(19) United States
(12) Patent Application Publication
(10) Pub. No.: US 2023/0397876 A1
(43) Pub. Date: Dec. 14, 2023

(54) SYSTEMS FOR ANALYZING PATTERNS IN ELECTRODERMAL ACTIVITY RECORDINGS OF PATIENTS TO PREDICT SEIZURE LIKELIHOOD AND METHODS OF USE THEREOF

(71) Applicant: The Children's Medical Center Corporation, Boston, MA (US)

(72) Inventors: Solveig VIELUF, Boston, MA (US); Tobias LODDENKEMPER, Boston, MA (US); Bo ZHANG, Boston, MA (US)

(73) Assignee: The Children's Medical Center Corporation, Boston, MA (US)

(21) Appl. No.: 18/236,602

(22) Filed: Aug. 22, 2023

Related U.S. Application Data
(63) Continuation of application No. PCT/US2022/017469, filed on Feb. 23, 2022.
(60) Provisional application No. 63/152,662, filed on Feb. 23, 2021.

(51) Int. Cl. A61B 5/00; A61B 5/0531; G16H 50/20; G16H 40/67

(52) U.S. Cl. CPC A61B 5/4094; A61B 5/0531; A61B 5/4857; A61B 5/7267; A61B 5/7275; A61B 5/746; G16H 50/20; G16H 40/67; A61B 5/681; A61B 5/0022

(57) ABSTRACT

Systems and methods of the present disclosure enable improved seizure detection and/or prediction using a seizure monitoring system. The system receives a data stream including wearable sensor data associated with a user, where the data stream includes electrodermal activity data and where the electrodermal activity data includes circadian rhythm-dependent amplitudes. The system receives a time associated with a seizure of the user. The system trains seizure machine learning model to identify a pre-ictal period associated with a time segment based on the circadian rhythm dependent amplitudes and the time associated with the seizure. The system deploys the seizure machine learning model to ingest a new data stream. Based on the new data stream, the seizure machine learning model predicts a seizure likelihood in a prediction period.

### CROSS REFERENCE TO RELATED APPLICATIONS

This application is a continuation under 35 U.S.C. § 111(a) of PCT International Patent Application No. PCT/US2022/017469, filed Feb. 23, 2022, designating the United States and published in English, which claims priority to and the benefit of U.S. Provisional Application No. 63/152,662 filed Feb. 23, 2021, the entire contents of each of which are incorporated by reference herein.

### BACKGROUND OF TECHNOLOGY

A better understanding of underlying causes, potential seizure patterns, and clinical implications are essential to improved seizure management and treatment algorithms. With recent developments in wearable technologies, further insight into autonomic manifestations of seizures may now be more attainable.

People with epilepsy exhibit suppressed vagal control and an autonomic imbalance toward increased sympathetic activity. Electrodermal activity (EDA), an autonomic marker for sympathetic skin activity, exhibits unique properties in the setting of seizures and thus can be used for seizure monitoring, as well as seizure detection and prediction. Biofeedback based on EDA for people with epilepsy (PWE) has decreased seizure likelihood in prior studies, and change may have been potentially related to central regulatory effects and modulation of EDA levels.

### SUMMARY OF DESCRIBED SUBJECT MATTER

Practical implementation of utilizing EDA to monitor seizures may be often challenged by low specificity of EDA changes. Potential factors contributing to the variability of EDA are environmental conditions, temperature, body position, and stress. In addition to these factors, diurnal rhythms are also likely an important source of variability within physiological signals. These 24-hour and circadian patterns are of relevance as they may contribute to rhythmic patterns of seizure occurrence and can thereby be related to the seizure-associated EDA response. Furthermore, time of seizure occurrence and type of seizure may also affect EDA peak height which varies within and between patients, particularly when controlled for a pre-ictal baseline. Therefore, a three-pronged approach is employed to elucidate the relationship of EDA, seizures, and diurnal patterns further.

The first aim of some embodiments may be to characterize 24-hour patterns in EDA, HR, and skin temperature (TEMP) recordings in patients with epilepsy, based on this central hypothesis that continuous EDA recordings show patterns of diurnal rhythms beyond the effects of thermoregulation.

Secondly, 24-hour patterns of EDA and TEMP are assessed to determine any difference between patients with and without seizures within the recording. In this setting, it may be hypothesized that the impact of seizures may have an effect on 24-hour modulation.

Thirdly, seizure-induced EDA response relative are evaluated to the EDA level and power calculated from a modulation pattern. In some embodiments, a seizure response within the EDA system may demonstrate an increase in EDA level and power following the seizure.

Day and nighttime patterns affect the dynamic modulation of brain and body functions and influence the autonomic nervous system response to seizures. Therefore, daytime and nighttime (“circadian” or “diurnal”) patterns of electrodermal activity (EDA) in patients with and without seizures may be evaluated.

In some embodiments, patients with 1) seizures (SZ), including focal impaired awareness seizures (FIAS) or generalized tonic-clonic seizures (GTCS), 2) no seizures and normal EEG (NEEG), or 3) no seizures but epileptiform activity in the EEG (EA) during video-EEG monitoring may be evaluated to determine EDA patterns. Patients may wear a device that continuously and/or periodically records EDA among other autonomic nervous system (ANS) metrics such as, e.g., temperature (TEMP). EDA levels, EDA spectral power, and/or TEMP levels may be analyzed. To investigate circadian patterns, a nonlinear mixed-effects model analysis may be performed. Relative mean pre-ictal (e.g., −30 min to seizure onset) and post-ictal (e.g., 30 min after seizure offset; e.g., 30 to 60 min after seizure offset) values may be compared for SZ subgroups.

### Definitions

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

As used herein, the term “data stream” refers to a periodic or continuous transmission of data from one system, device, or component to another via any suitable wired or wireless data communication devices and techniques.

As used herein, the term “tonic clonic seizure” refers to a type of seizure, also known as a grand mal seizure, characterized by a tonic phase where the body becomes rigid, followed by a clonic phased where the body undergoes uncontrolled jerking.

As used herein, the term “ictal” refers to the period a physiologic state or event such as a seizure and may be used to further indicate the period of a, e.g., stroke, headache, inflammation, flare-up, mental health episode, or in general any relapsing-remitting diseases.

As used herein, the term “preictal” refers to the time period preceding an ictal event of variable duration.

As used herein, the term “interictal” refers to the period between ictal events.

As used herein, the term “postictal” refers to the period refers to the state shortly after an ictal event.

As used herein, the term “periictal” refers to the period encompassing preictal, ictal and postictal periods.

As used herein, the term “electrodermal activity” refers to a measure of neurally mediated effects on sweat gland permeability, observed as changes in the resistance of the skin to a small electrical current, or as differences in the electrical potential between different parts of the skin.

As used herein, the term “integration window” refers to a time period including data on which an operation may be to be performed.

As used herein, the term “ground-truth” refers to one or more sets of object, provable data.

As used herein, the term “multi-modal” refers to device properties or an analysis that combines multiple signals that relate to the activity of different autonomic systems.

As used herein, the term “electroencephalography (EEG)” refers to the measurement of electrical activity in different parts of the brain.

As used herein, the term “electrocorticography (ECoG)” refers to the direct recording of electrical potentials associated with brain activity from the cerebral cortex.

As used herein, the term “electrocardiography (ECG)” refers to the measurement of electrical activity in the heart using electrodes placed on the skin of the limbs and chest.

As used herein, the term “biosensor” refers to a device configured to and/or capable of producing data streams of clinical, biological or physiological parameters by sensing such parameters from a patient.

As used herein, the term “sensitivity” refers to the true positive seizure prediction rate.

As used herein, the term “time in warning” refers to the fraction of time spent in warning.

As used herein, the term “improvement over chance” refers to. to enhanced accuracy, sensitivity, and specificity comparing our model to random guess classification.

As used herein, the term “based on” may be not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “real-time” may be directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc. It may be understood that at least one aspect or functionality of various embodiments described herein can be performed in real-time or dynamically, or both.

As used herein, the terms “computer engine” and “engine” identify at least one software component and/or a combination of at least one software component and at least one hardware component which are designed, programmed or otherwise configured to manage or control other software and hardware components (such as the libraries, software development kits (SDKs), objects, etc.).

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but may be not limited to, a mobile phone, Personal Digital Assistant (PDA), Blackberry™, Pager, Smartphone, or any other reasonable mobile electronic device.

As used herein, terms “proximity detection,” “locating,” “location data,” “location information,” and “location tracking” refer to any form of location tracking technology or locating method that can be used to provide a location of, for example, a particular computing device, system or platform of the present disclosure and any associated computing devices, based at least in part on one or more of the following techniques and devices, without limitation: accelerometer(s), gyroscope(s), Global Positioning Systems (GPS); GPS accessed using Bluetooth™; GPS accessed using any reasonable form of wireless and non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this may be in no way meant to be a limitation.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set may be to be interpreted as meaning each of the items singularly as alternatives, as well as any combination of the listed items.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 depicts a block diagram of an exemplary computer-based system and platform for epilepsy monitoring and risk prediction in accordance with one or more embodiments of the present disclosure.

FIG. 2 illustrates one 20-hour exemplary recording of EDA level, EDA power and temperature recorded at wrist in a first example embodiment according to aspects of the present disclosure.

FIG. 3A illustrates example ANS measurements for a whole patient group in a first example embodiment according to aspects of the present disclosure, where a top row shows mean and standard error for the whole patient group for EDA level, EDA power, and TEMP level (from left to right) and a lower level shows the best fitting 24-hour pattern from nonlinear mixed-effects model analysis for EDA level, EDA power, and TEMP level (from left to right).

FIG. 3B illustrates example patient group-specific ANS measurements in a first example embodiment according to aspects of the present disclosure, where group specific mean values (top row) and fitted 24-hour patterns (lower row) show EDA level, EDA power, and TEMP level (from left to right) for the seizure (SZ), the normal EEG (NEEG), and the epileptiform activity (EA) groups.

FIG. 4A shows an example 24-hour pattern for the SZ group in a first example embodiment according to aspects of the present disclosure, where for the left side shows EDA level and the right size shows power.

FIG. 4B shows an example EDA signal in peri-ictal period in relation to expected values for the SZ group in a first example embodiment according to aspects of the present disclosure, where for the left side shows EDA level and the right size shows power.

FIG. 4C depicts example summarized median and standard error of EDA levels (left) and EDA power (right) in pre-ictal, post-ictal I and post-ictal II periods separated for GTCS and FIAS in a first example embodiment according to aspects of the present disclosure.

FIG. 5 depicts a schematic illustration of data collection and analysis steps in a second example embodiment according to aspects of the present disclosure.

FIG. 6 depicts individual recordings of EDA, TEMP, HR (from top to bottom) averaged over 10-min segments of no-seizure (left panel) and seizure patients (middle panel) are displayed over 24 hours in a second example embodiment according to aspects of the present disclosure.

FIG. 7 depicts an example within patient comparison of ANS measurements in the second example embodiment according to aspects of the present disclosure.

FIG. 8 depicts a schematic illustration of the experiment setup in a third example embodiment according to aspects of the present disclosure.

FIG. 9 depicts an inclusion diagram that visualizes the patient and signal selection procedures in a third example embodiment according to aspects of the present disclosure.

FIG. 10 depicts a number of patients having a seizure at a time of the day in a third example embodiment according to aspects of the present disclosure.

FIG. 11 depicts a block diagram of an exemplary computer-based system and platform 1100 in accordance with one or more embodiments of the present disclosure.

FIG. 12 depicts a block diagram of another exemplary computer-based system and platform 1200 in accordance with one or more embodiments of the present disclosure.

FIG. 13 depicts illustrative schematics of an exemplary implementation of the cloud computing/architecture(s) in which embodiments of a system for epilepsy monitoring and risk prediction may be specifically configured to operate in accordance with some embodiments of the present disclosure.

FIG. 14 depicts illustrative schematics of another exemplary implementation of the cloud computing/architecture(s) in which embodiments of a system for epilepsy monitoring and risk prediction may be specifically configured to operate in accordance with some embodiments of the present disclosure.

### DETAILED DESCRIPTION

Various detailed embodiments of the present disclosure, taken in conjunction with the accompanying figures, are disclosed herein; however, it may be to be understood that the disclosed embodiments are merely illustrative. In addition, each of the examples given in connection with the various embodiments of the present disclosure may be intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” may be not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set may be to be interpreted as meaning each of the items singularly as alternatives, as well as any combination of the listed items.

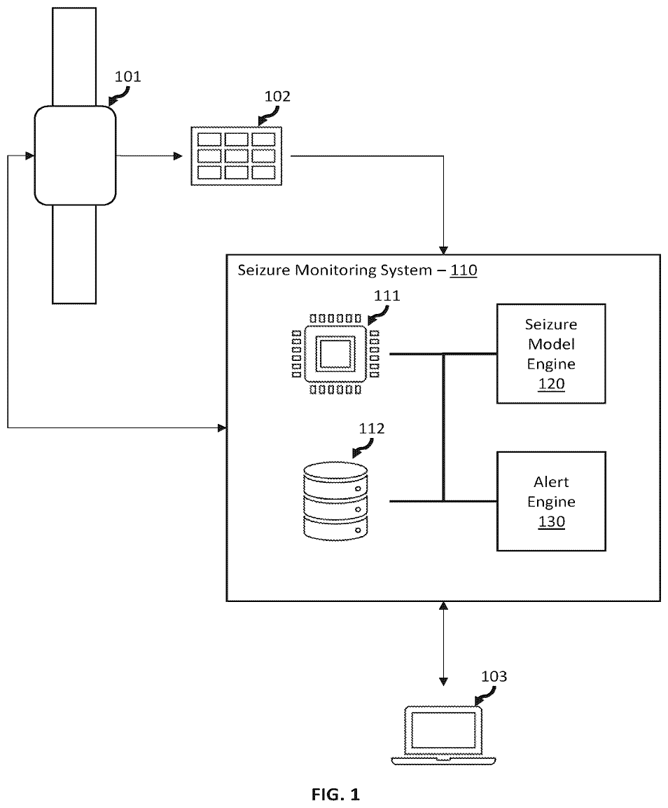

FIGS. 1 through 14 illustrate systems and methods of seizure monitoring, detection and prediction, as well as forecasting. The following embodiments provide technical solutions and technical improvements that overcome technical problems, drawbacks and/or deficiencies in the technical fields involving seizure monitoring that relies on intrusive and computationally inefficient monitoring of patient activity via video and/or EEG data. Both video and EEG require relative large and complicated devices that cannot be feasible worn on a regular basis, and may also output complex and extensive data that requires a large amount of memory, storage and processing resources to analyze. As explained in more detail, below, technical solutions and technical improvements herein include aspects of improved seizure monitoring, detection and/or prediction by leveraging circadian/diurnal patterns in ANS activity, which may be detected with a lightweight and efficient wearable device for detection and/or prediction of seizure risk/likelihood for advance alert and recommendation of increased monitoring and/or intervention in an efficient and practical manner. Based on such technical features, further technical benefits become available to users and operators of these systems and methods. Moreover, various practical applications of the disclosed technology are also described, which provide further practical benefits to users and operators that are also new and useful improvements in the art.

Currently, the standard treatment outcome measure in epilepsy is seizure reduction or prevention. Therefore, effective epilepsy treatment relies on determining seizure frequency, and ideally preemptive seizure likelihood assessment. Seizure diaries are widely used for seizure tracking and show potential for seizure forecasting based on cyclic patterns. Furthermore, seizure likelihood assessments have been accomplished in adults with neuro-surgically implanted intracranial electrodes, indicating that detectable neuro-physiological changes precede seizures. However, a less invasive seizure forecasting method based on passively recorded data is needed.

A better understanding of underlying causes, potential seizure patterns, and clinical implications are essential to improved seizure management and treatment algorithms. With recent developments in wearable technologies, further insight into autonomic manifestations of seizures may now be more attainable. Predicting a risk of or likelihood of a future seizure may provide information useable to make recommendations for seizure monitoring.

People with epilepsy exhibit suppressed vagal control and an autonomic imbalance toward increased sympathetic activity. Electrodermal activity (EDA), an autonomic marker for sympathetic skin activity, exhibits unique properties in the setting of seizures and thus can be used for seizure monitoring, as well as seizure detection and prediction. Biofeedback based on EDA for people with epilepsy (PWE) has decreased seizure likelihood in prior studies, and change may have been potentially related to central regulatory effects and modulation of EDA levels.

In some embodiments, wearable technologies may enable improved seizure tracking based on autonomic manifestations of seizures. Autonomic nervous system (ANS) changes occur frequently in children with epilepsy and may serve as a potential biomarker for seizure risk. Specifically, EDA, an autonomic marker for sympathetic skin activity, exhibits unique properties in the setting of seizures and thus may be used to determine seizure likelihood, either alone or when combined with other ANS modalities and potentially additional clinical information.

Predicting seizure risk may be facilitated using wearable recordings measuring autonomic nervous system (ANS) activity. Retrospectively analyzing self-reported seizures shows a potential to estimate seizure likelihood in the future based on seizure cycles. If wearable recordings contain relevant information in the seizure diaries and wearable recordings may jointly be valuable seizure risk assessment tools. Analysis of 24-hour ANS data, specifically electrodermal activity, suggests larger differences between patients with and without seizures at night than during the day, suggesting that short-term recordings before going to bed and after waking up may provide predictive information.

Jointly with clinical data, analysis of short-time-window ANS activity recordings may be used to determine seizure likelihood for the following day and thereby inform the following clinical decisions: hospital admission scheduling, hospital stay extension, and deployment of ambulatory seizure monitoring equipment in the patient's home, and timing of anti-seizure medication or management adjustment, or risk assessment regarding activities during the following day (e.g., the ability to partake in activities otherwise precluded during high risk seizure periods including driving, etc.). Despite the great potential of wearables, the algorithm development and implementation, acceptance, and compliance to continuously wear the devices remains a challenge. Performing daily, brief measurements could make seizure risk assessment more accessible to patients and could be a starting point for developing reliable seizure prediction systems. For example, patients could record short measurements once per day while taking medications.

Practical implementation of utilizing EDA to monitor seizures may be often challenged by low specificity of EDA changes. Potential factors contributing to the variability of EDA are environmental conditions, temperature, body position, and stress. In addition to these factors, diurnal rhythms are also likely an important source of variability within physiological signals. These 24-hour and circadian patterns are of relevance as they may contribute to rhythmic patterns of seizure occurrence and can thereby be related to the seizure-associated EDA response. Furthermore, time of seizure occurrence and type of seizure may also affect EDA peak height which varies within and between patients, particularly when controlled for a pre-ictal baseline. Therefore, a three-pronged approach is employed to elucidate the relationship of EDA, seizures, and diurnal patterns further.

In some embodiments, system(s) and/or method(s) of the present disclosure may be to characterize patterns in EDA, skin temperature (TEMP), or other ANS measurements or any combination thereof in patients with epilepsy, based on the central hypothesis that continuous EDA recordings show patterns of diurnal rhythms beyond the effects of thermoregulation.

In some embodiments, 24-hour patterns of EDA and TEMP may be used to determine a difference between patients with and without seizures within the recording. In this setting, it may be hypothesized that the impact of seizures may have an effect on 24-hour modulation.

In some embodiments, short-term or momentary measurements may be used to characterize patterns of EDA, HR, and TEMP to determine a difference between patients with and without seizures within the recording. In some embodiments, the measurements may be performed for a predetermine duration at predetermined time points during a patient's circadian cycle, such as, e.g., for a 5 minute period, 10 minute period, 15 minute period, 20 minute period, 25 minute period, 30 minute period, one hour period, or other suitable duration of time in a range of between 1 minute and 8 hours or any other suitable range. In some embodiments, the measurements may be recorded at least once during a nighttime period and at least once during a daytime period to characterize the diurnal rhythms. In some embodiments, the nighttime period and the daytime period may be predetermined to be the same times every day, such as, e.g., 9:00 PM every night and 6:00 AM every morning, or any other suitable time when a patient is going to sleep and when a patient is waking up.

In some embodiments, seizure-induced EDA responses are evaluated relative to the EDA level and power calculated from a modulation pattern. In some embodiments, a seizure response within the EDA system that may demonstrate an increase in EDA level and power following the seizure.

FIG. 1 illustrates a block diagram of an exemplary computer-based system and platform for seizure monitoring and seizure risk prediction in accordance with one or more embodiments of the present disclosure.

In some embodiments, a seizure monitoring system 110 using wearable sensor data 102 to forecast seizure risks in a user using EDA and/or TEMP and in combination with HR measurements without the need for expensive and cumbersome EEG, EKG and ECOG tests that would ordinarily be used for assessing seizures. The wearable sensor data 102 can be provided to the seizure monitoring system 110 from a wearable sensor 101 as a continuous stream of data. Thus, the seizure monitoring system 110 may monitor the user's sensor data 102 in real-time, thus enabling timely intervention or mitigation of impending seizures using discrete, wearable sensing devices. As a result, the seizure monitoring system 110 uses peripheral signals from a device having a compact, wearable design that may limit the risk of stigmatization, affords more easy application, and may altogether increase patient adherence relevant for long-term ambulatory use, while also providing effective, real-time monitoring beyond the typical occasional and expensive EEG, ECG and ECOG testing.

As such, in some embodiments, the wearable sensor 101 can include any suitable sensing device for sensing physiological parameters, such as ANS parameters including, e.g., EDA, TEMP, HR, or any other suitable ANS measurement or any combination thereof. For example, the wearable sensor 101 can include a device for sensing parameters, such as, e.g., electrodermal activity, body temperature, blood volume pulse, heart rate, heart rate variability, blood oxygen content, blood glucose data, electrocardiographic data and actigraphy (accelerometer-based and location-based activity data), electroencephalogram measurements, time, date, medications, self-reported seizures, or any combination thereof. In some embodiments, the wearable sensor 101 can include, e.g., a smartwatch, a wristband sensor, a chest strap, a smart ring, or other health tracking sensor device, and combinations thereof. The wearable sensor data 102 may be collected for a suitable sample period at a sampling rate, e.g., at about 60 hertz (Hz), 30 Hz, 20 Hz, 15 Hz, 10 Hz, 5 Hz, 1 Hz or other sampling rate. Thus, the seizure monitoring system 110 may be provided with streams of each physiological parameter in the wearable sensor data 102 for each of a suitable sample period to characterize a stage in a circadian cycle.

In some embodiments, the sample period may be, e.g., a 5 minute period, 10 minute period, 15 minute period, 20 minute period, 25 minute period, 30 minute period, one hour period, or other suitable duration of time in a range of between 1 minute and 8 hours or any other suitable range. In some embodiments, the sample period may be established as one or more sample periods in each stage of the circadian cycle. For example, the circadian cycle may include a sleep stage and a wake stage, however other stages may be included, such as, e.g., early sleep stage, mid sleep stage, late sleep stage, early wake stage, mid wake stage, late wake stage, among others or any combination thereof. In some embodiments, the sample periods for a given patient may be established according to predetermined times of the day associated with a circadian cycle, e.g., for an average circadian cycle, or may be customized for the patient based on the patient's sleep-wake patterns. For example, the sample periods may include a sleep sample period associated with a sleep stage, e.g., at 8:00 PM, 8:15 PM, 8:30 PM, 8:45 PM, 9:00 PM, 9:15 PM, 9:30 PM, 9:45 PM, 10:00 PM, 10:15 PM, 10:30 PM, 10:45 PM, 11:00 PM, 11:15 PM, 11:30 PM, 11:45 PM, 12:00 AM, 12:15 AM, 12:30 AM, 12:45 AM, or other suitable sample period associated with a sleep stage or multiple sample periods. For example, the sample periods may include a wake sample period associated with a wake stage, e.g., at 5:00 AM, 5:15 AM, 5:30 AM, 5:45 AM, 6:00 AM, 6:15 AM, 6:30 AM, 6:45 AM, 7:00 AM, 7:15 AM, 7:30 AM, 7:45 AM, 8:00 AM, 8:15 AM, 8:30 AM, 8:45 AM, 9:00 AM, 9:15 AM, 9:30 AM, 9:45 AM, or other suitable sample period associated with a sleep stage or multiple sample periods.

In some embodiments, the sleep sample period may include continuous measurements of one or more ANS parameters throughout the sleep stage. In some embodiments, to reduce computational and power resource requirements associated with continuous measurement, the sleep sample period may include one or more sleep sample periods of shorter duration than the sleep stage, such as, e.g., one or more sample periods intermittently or at predetermined times throughout the sleep stage. In some embodiments, the sample period may include a single sleep sample period at the beginning of the sleep stage, such as at 9:00 PM.

In some embodiments, the wake sample period may include continuous measurements of one or more ANS parameters throughout the wake stage. In some embodiments, to reduce computational and power resource requirements associated with continuous measurement, the wake sample period may include one or more wake sample periods of shorter duration than the wake stage, such as, e.g., one or more sample periods intermittently or at predetermined times throughout the wake stage. In some embodiments, the sample period may include a single wake sample period at the beginning of the wake stage, such as at 6:00 AM.

In some embodiments, the seizure monitoring system 110 may receive the wearable sensor data 102 as a data stream and/or batch of data of each of the physiological parameters. In some embodiments, the seizure monitoring system 110 may use a combination of software and hardware components to record and process the data to forecast a risk of the user experiencing a seizure and generate an alert to the user indicating the risk.

Examples of software components may include programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment may be implemented using hardware elements and/or software elements may vary in accordance with any number of factors, such as desired computational rate, power levels, heat tolerances, processing cycle budget, input data rates, output data rates, memory resources, data bus speeds and other design or performance constraints.

Examples of hardware components may include processors 111, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

In some embodiments, the hardware components may also include a data storage 112. In some embodiments, the data storage 112 may include, e.g., a suitable memory or storage solutions for providing electronic data to the seizure monitoring system 110. For example, the data storage 112 may include, e.g., a centralized or distributed database, cloud storage platform, decentralized system, server or server system, among other storage systems, or the data storage 112 may include, e.g., a hard drive, solid-state drive, flash drive, or other suitable storage device, or the data storage 112 may include, e.g., a random access memory, cache, buffer, or other suitable memory device, or any other data storage solution and combinations thereof.

In some embodiments, the data storage 112 may receive and record the continuous stream and/or batch of wearable sensor data 102 among other patient data, including electronic medical record data, clinical data, radiological and other imagery and test results, medication and medication dosages, and any other health-related data, such as any data from an electronic medical health record or other health record. The wearable sensor data 102 may be accessible by, e.g., a seizure model engine 120 and an alert engine 130, e.g., via the processor 111. However, in some embodiments, the wearable sensor data 102 may be provided directly to seizure model engine 120 and the alert engine 130 before or instead of being stored in the data storage 112.

In some embodiments, the seizure model engine 120 includes a combination of hardware and/or software for predicting seizure risk at a given time based on the wearable sensor data 102, or a subset of the wearable sensor data 102 pertaining to a selected segment of time preceding the given time. In some embodiments, the seizure model engine 120 may model biological parameter activity (e.g., EDA measurements, TEMP measurements, or other suitable ANS measurement or any combination thereof) to model epilepsy and seizure patterns in any given prediction period, and to predict a seizure risk level once every prediction period. In some embodiments, the prediction period may be, e.g., every minutes, every hour, every 24 hours, every day, or every night or both, a coming sleep period, a coming wake period, or other suitable period. Thus, for each prediction period, the seizure model engine 120 may instantiate a seizure risk model and develop seizure risk prediction for that prediction period based on the wearable sensor data 102 and other health-related data associated with the prediction period. In some embodiments, the prediction periods are continuous, non-overlapping segments of time including the wearable sensor data 102 during that time.

However, in some embodiments, the prediction periods may overlap, such that the time segment preceding the given time at which a seizure risk prediction may be made overlaps with a previous time segment for predicting seizure risk at a previous time. For example, a first 1, 2, 3, 4, 6, hour prediction period may include the time segment from t=0 hours to t=1, 2, 3, 4, or 6 hours for a prediction at t=1, 2, 3, 4, or 6 of seizure risk, with a second 1, 2, 3, 4, or 6 hour prediction period including the time segment from t=1, 2, 3, 4, or 6 hours to t=7, 8, 9, 10 or 12 hours for a prediction at t=7, 8, 9, 10 or 12 hours of seizure risk. Thus, the prediction period may include a moving time window approach that may form predictions based on windows having a size according to the prediction period and move according to, e.g., an update period. In some embodiments, the update period may be any suitable increment of time less than the prediction period.

In some embodiments, the seizure model engine 120 may include, e.g., a suitable machine learning algorithm for classifying a seizure risk based on the physiological parameters of the wearable sensor data 102 and health-related data, such as electronic medical record data. The classification of seizure risk can include a period type relative to a seizure, or ictal period. For example, the seizure model engine 120 may forecast a seizure risk based on a classification of any given time period as, e.g., preictal, postictal or interictal.

Thus, in some embodiments, the prediction based on the wearable sensor data 102 for a particular prediction period results in a seizure risk forecast including the classification of that the prediction period as preictal, postictal or interictal. However, in some embodiments, since postictal periods follow a seizure event, the predictive power of such periods are low. Thus, the seizure model engine 120 may be configured to predict whether a prediction period may be preictal or not preictal (e.g., interictal and/or postictal). For example, the seizure model engine 120 may be trained to recognize physiological parameters during a time period that would indicate that preictal period correspond to a seizure being imminent within, e.g., about 61 minutes of a current time.

Thus, in some embodiments, the seizure model engine 120 may include a machine learning model for differentiating between the periictal periods, and in particular, in distinguishing a preictal period indicating a high risk of an impending seizure, e.g., a high risk of a seizure occurring within a seizure occurrence period, e.g., within about 61 minutes of the high risk indication. In some embodiments, a data segment may be preictal if it occurs between 61 minutes and 1 minute prior to a seizure, thus leaving a one-minute buffer prior to seizure onset. This preictal window definition may be commensurate with other seizure forecasting research using EEG and ECoG and may account for potential small ambiguities in determining the exact seizure onset between EEG and wristband. A data segments may be classified as interictal or not preictal to indicate that the associated prediction period may be two hours or more away from any seizure.

To do so, in some embodiments, the seizure model engine 120 may include artificial intelligence (AI) or machine learning techniques for forecasting a preictal period based on wearable sensor data 102 of physiological parameters and physiological data from, e.g., electronic medical health records, the techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net), long short-term memory network or other suitable network. In some embodiments and, optionally, in combination of any embodiment described above or below, an exemplary implementation of Neural Network may be executed as follows:
i) define Neural Network architecture/model,
ii) transfer the input data to the exemplary neural network model,
iii) train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values, functions and aggregation functions. For example, an activation function of a node may be a step function, sine function, continuous or piecewise linear function, sigmoid function, hyperbolic tangent function, or other type of mathematical function that represents a threshold at which the node may be activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the wearable sensor 101 may be positioned, e.g., on a wrist and/or ankle during video-EEG to generate training and/or validation data sets for producing a trained machine learning algorithm for use by the seizure model engine 120 to detect and/or predict seizure likelihood based on wearable sensor data 102. In some embodiments, the training and/or validation data may include patients with no seizures, at least one generalized tonic-clonic seizure (GTCS), and/or at least one focal impaired awareness seizure (FIAS). In some embodiments, the training and/or validation data may exclude patients with status epilepticus (seizures longer than 5 or 10 minutes for GTCS or FIAS, respectively) and/or with only other seizure types during the recording. In some embodiments, where multiple recordings are available for a given patient, the earliest recording with seizures may be included. Where recordings from both body sides are available, the left may be selected over the right body side, assuming to have mostly right-handers and consequently signal quality to be higher for the non-dominant hand.

In some embodiments, to curate the wearable sensor data 102, recordings from patients may be include the following signals: EDA at a suitable sampling rate (e.g., 1, 2, 3, 4, 5, 6, 7, 8, 9, 10, 11, 12, etc. Hz), peripheral body temperature (TEMP) at a suitable sampling rate (e.g., 1, 2, 3, 4, 5, 6, 7, 8, 9, 10, 11, 12, etc. Hz), and heart rate (HR) at a suitable sampling rate (e.g., 1, 2, 3, 4, 5, 6, 7, 8, 9, 10, 11, 12, etc. Hz) among other ANS measurement recordings. In some embodiments, the root mean square of successive differences (RMSSD) may be calculated from the inter-beat interval (IBI). In some embodiments, the training and/or validation data sets may be pruned to include two sets of recordings of wearable sensor data 102 in the evening and in the morning.

In some embodiments, the clinical data may be added to the wearable sensor data 102. In some embodiments, the clinical data may include, e.g., chart review of clinical notes, e.g., according to a standardized clinical data acquisition tool. In some embodiments, the following variables may be collected and stored: sex, age during enrollment, age at first seizure, MRI finding, anti-seizure medication (ASM) reduction during stay, generalized slowing on interictal EEG, interictal spikes, and seizure before measurement during the same recording. In some embodiments, to create the training and/or validation data, a board-certified epileptologist may review the video-EEG recordings to determine seizure type and electrographic onset and offset times. GTCS included tonic-clonic seizures of focal and generalized onset.

In some embodiments, differences in the wearable sensor data 102 recordings between patients with and without impending seizures may be analyzed to construct the training data set. Group assignment could be different for individuals depending on seizure times, for example, a patient having a seizure at 11 p.m. would be in the seizure group for the evening recording but the without-seizure group for the morning recording, as no seizure was impending. In some embodiments, group differences of mean EDA, TEMP, HR, and RMSSD between the with seizure and without seizure groups may be analyzed (e.g., inclusion in seizure group determined by the patient having a seizure after 9 p.m. or after 6 a.m., for the two analyses respectively) via, for example, Mann-Whitney U tests.

In some embodiments, for group classification, the seizure model engine 120 may implement one or more machine learning algorithms. Moreover, the machine learning algorithms may be evaluated for performance, such as each of seven machine learning algorithms: logistic regression (LR), k-nearest neighbor (KNN), random forest (RF), adaptive boosting (AB), Gaussian naive Bayes (GNB), and support vector machine (SVM) with linear or radial basis function kernel classifiers. In some embodiments, cross-validation may be used, such as, e.g., 10-fold cross-validation, in which hyperparameters are tuned by recursively by splitting the training dataset 10 times.

In some embodiments, for feature selection, the training and/or validation data sets may include a seizure during recording since the wearable sensor 101 was put on but before analysis time (e.g., time from the wearable sensor data 102 recording start to 9 p.m. or 6 a.m. respectively), EDA, HR, RMSSD, sex, age at first seizure, MRI findings, interictal generalized slowing (yes/no), interictal spikes (yes/no), and anti-seizure medication (ASM) reduction (yes/no). In some embodiments, the features may be input into the machine learning algorithm(s) of the seizure model engine 120 to learn a correlation between the wearable sensor data 102 and/or differences in the wearable sensor data 102 across the circadian cycle to the seizure classifications identified by the epileptologists.

In some embodiments, the seizure model engine 120 processes the features by applying the parameters of the machine learning algorithm(s) to produce a model output vector. In some embodiments, the model output vector may be decoded to generate one or more labels indicative of seizure risk and/or seizure likelihood within a prediction period, such as at a current time and/or at in a coming period. In some embodiments, the model output vector may include or may be decoded to reveal a numerical output, e.g., one or more probability values between 0 and 1 where each probability value indicates a degree of probability that a particular label correctly classifies the wearable sensor data 102. In some embodiments, the seizure model engine 120 may test each probability value against a respective probability threshold. In some embodiments, each probability value has an independently learned and/or configured probability threshold. Alternatively, or additionally, in some embodiments, one or more of the probability values of the model output vector may share a common probability threshold. In some embodiments, where a probability value is greater than the corresponding probability threshold, the wearable sensor data 102 is labeled according to the corresponding label. For example, the probability threshold can be, e.g., greater than 0.5, greater than 0.6, greater than 0.7, greater than 0.8, greater than 0.9, or other suitable threshold value. Therefore, in some embodiments, the seizure model engine 120 may produce the seizure risk and/or likelihood for a particular wearable sensor data 102 recording based on the probability value(s) of the model output vector and the probability threshold(s).

In some embodiments, the parameters of the machine learning algorithm(s) may be trained based on known outputs. For example, the wearable sensor data 102 may be paired with a target classification or known classification (e.g., based on the seizure labels specified by the epileptologist) to form a training pair. In some embodiments, the wearable sensor data 102 may be provided to the seizure model engine 120, e.g., encoded in a feature vector, to produce a predicted label. In some embodiments, an optimizer associated with the seizure model engine 120 may then compare the predicted label with the known output of a training pair to determine an error of the predicted label. In some embodiments, the optimizer may employ a loss function, such as, e.g., Hinge Loss, Multi-class SVM Loss, Cross Entropy Loss, Negative Log Likelihood, or other suitable classification loss function to determine the error of the predicted label based on the known output.

In some embodiments, model performance may be evaluated by randomly shuffling the data labels k times based on k-fold cross-validation and compare classifier performance of the shuffled and original labels, e.g., with a t-test. For LR, KNN, RF, AB, GNB, and SVM (linear and rbf) classifiers, providing patient-specific probability scores, performance may be evaluated with area under the curve of receiver operating characteristic (AUC-ROC) and with the Brier score, among other measures.

In some embodiments, based on the training, later wearable sensor data 102 may be provided to the seizure model engine 120. Accordingly, based on the wearable sensor data 102, e.g., including recordings across the circadian cycle, may be used to create input features, alone and/or with additional biomarkers and/or clinical data, to the machine learning model(s) of the seizure model engine 120. The seizure model engine 120 may then output a classifications for the prediction period as a high risk or high likelihood seizure period, or a low risk or low likelihood seizure period.

In some embodiments, the classification from the seizure model engine 120 can be provided to an alert engine 130 for generating an alert that a seizure event may be likely during the prediction period based on the prediction period being classified as a high-risk period. In some embodiments, the seizure model engine 120 may first provide each seizure classification for each prediction period to the data storage 112 to record the prediction period with an indication of the associated classification. In some embodiments, the classification may be provided to the data storage 112, which may then be accessed by the alert engine 130, or the seizure model engine 120 may provide the classification directly to the alert engine 130, either before, after or concurrently with recording the classification in the data storage 112.

In some embodiments, the classification from the seizure model engine 120 can include a binary classification (e.g., high-risk or low-risk). The binary classification can include a probability that a given prediction period may be a high-risk period based on the wearable sensor data 102. For example, the classification may include a numerical value on a scale from 0 to 1, where 0 indicates a zero percent probability of the prediction period being a high-risk period, and where 1 indicates a one hundred percent probability of the prediction period being a high-risk period. In practice, any given prediction period may be unlikely to be a 0 or a 1, but likely may be classified somewhere in between.

In some embodiments, the alert engine 130 may determine that the probability of the classification indicates a high-risk period using, e.g., a risk threshold. For example, where the probability rises above a risk threshold of, e.g., 0.5, 0.52, 0.54, 0.56, 0.58, 0.6, the alert engine 130 may determine that the prediction period may be a high-risk period, thus indicating that a seizure may be likely within the period, such as, e.g., within about an hour or within any amount of time within the period. Thus, the alert engine 130 may generate an alert to the user. In some embodiments, the probability for each prediction period may be compared to the risk threshold.

However, irregularities may occur at any particular prediction period that may give rise to a high probability of the high-risk period classification for one prediction period. Thus, a seizure may only be actually imminent where the physiological parameters consistently indicate a high-risk period according to the associated classification probabilities. Therefore, in some embodiments, an integration window may be employed where the high-risk classification probabilities for each prediction period within a windowed time span may be aggregated and then compared to the risk threshold. For example, the integration window may encompass a time span.

In some embodiments, the alert generator 130 may generate an alert including, e.g., a visual indication via a graphical user interface, an audible indication, a vibration or tactile indication, or other alert notifying the user of the risk of a seizure based on the high-risk classification. In some embodiments, the alert may be provided to a user computing device 103 or to the wearable sensor 101, or both. In some embodiments, the user computing device 103 may include, e.g., a personal computer, mobile device, wearable device, tablet computer, or other computing device associated with the user. For example, the user computing device 103 and/or the wearable sensor 101 may display the visual indication and/or emit the audible, vibration and/or tactile indication such that the user may perceive the alert of the risk of an imminent seizure. As a result, the user may receive a real-time warning for imminent seizures, enabling the user to take preventative or mitigating steps to avoid harm that may result from a seizure. Similarly, the user may receive a real-time indication that seizure risk may be low, a real-time indication of the current seizure risk at any time, or other real-time seizure risk indication techniques. The alert generator 130 may also be configured to determine a mitigation or treatment strategy along with the alert, such as, e.g., a notification to stop a car, lie down, ingest a prescribed medication, etc. The alert generator 130 may also be embedded in a closed-loop setup linked to a device to administer treatment and thereby lower the risk of a seizure or prevent it completely. This treatment device may include a system to apply a fast-acting antiseizure medication or a neuromodulatory device which, for example, administers electrical stimulation to the brain in order to decrease seizure risk, apply real-time seizure detection solutions (e.g., EEG monitoring, video monitoring, etc.), among other mitigations, interventions and/or observations or any combination thereof.

### Example Embodiment 1

### Materials and Methods

In some embodiments, in a first example embodiments, patients may be provided with the wearable sensor 101 such as a wearable biosensor including, e.g., a wrist- or ankle-worn sensor such as, e.g., an Empatica E4 biosensor, an Apple Watch, a Samsung Galaxy Watch, a FitBit, or other suitable smartwatch, health tracker or biometric tracking wearable or any combination thereof. In some embodiment, the wearable sensor 101 may provide ANS measurements to a seizure monitoring system 110 to use machine learning to predict whether a coming period is of high risk or low risk for a seizure. In some embodiments, to train and validate the machine learning model, the patients may be monitored concurrently with video-EEG (vEEG) monitoring. In some embodiments, for ground-truth data, clinical epileptologists may review the video-EEG recordings to determine clinical seizure type based on the ILAE classification, including EEG localization and onset and offset times.

In some embodiments, patients may be evaluated to assess the seizure monitoring system 110 and train the seizure model engine 120. To perform such an assessment, patients may be evaluated where the patients have at least 8 hours of wearable sensor data 102 and qualify for one of the following three subgroups may be selected: a) patients who had at least one seizure during vEEG (GTCS or FIAS, or other seizure types) while wearing the wearable sensor 101 (SZ), b) patients with normal EEG (NEEG) during vEEG (no seizures, interictal epileptiform activity or encephalopathic patterns), and c) patients with epileptiform activity but no seizures during vEEG (EA). For additional sub-analyses of seizure-related EDA response relative to the 24-hour modulated SZ group values, seizures from patients of the initial cohort may be selected who have at least one seizure during vEEG (GTCS or FIAS) while wearing the wearable sensor 101, regardless of the total time recorded. For training and/or validation, more than one seizure per patient may be used in some cases (patients with: 1 seizure=23, 2 seizures=6, 3 seizures=5, 4 seizures=1, 5 seizures=1). Patients with low quality signal in pre- or post-ictal periods (e.g., no EEG or video-EEG available) patients with status epilepticus, and patients that do not have a seizure-free interval between successive seizures for a minimum period of time may be excluded, such as for a minimum period of time including, e.g., 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, or any other suitable minimum period in the range of 1 minutes and 300 minutes.

Data Recording and Quality Check

In the first example embodiment, the patients may wear the wearable sensor 101 for long-term recording. In some embodiments, the wearable sensor 101 may capture EDA with a sampling rate, e.g., of 4 Hz or other suitable sampling rate. The recordings may start between 9 a.m. and 4 p.m. Patients with low signal quality, recordings with less than 8 hours of data, and patients who do not have a seizure-free interval of the minimum period during the recording may be excluded. To identify unreliable signals or biosensor removal, the remaining data may be screened for low EDA activity (e.g., <0.001 for more than 15 minutes) and sudden temperature changes (e.g., >3° C.). In these cases, the recording may be analyzed from that point forward or until that point, and previous or subsequent samples may be discarded to preserve signal quality. If there may be multiple periods of concern for disconnection or artifact throughout the measurement, the patient may be excluded from analysis.

In some embodiments, the mean EDA amplitude of the sections that may be included in the 24-hour period may be calculated and compared the EDA levels recorded from the wearable sensor 101 recordings from the wrist (mean=1.25 μS, SD=1.53 μS) and ankle (mean=1.03 μS, SD=1.69 μS). As there may be no difference (T=0.73, p=0.470), the patients may be combined. For the SZ group, signal quality may be also screened for a pre-ictal period, such as, e.g., 30 minutes of pre-ictal data, and a post-ictal period, such as, e.g., 60 minutes of post-ictal data. In some embodiments, seizures may be excluded when one or more of the following conditions may be observed: low EDA activity (<0.001 μS) for more than 5 minutes, or repetitively (>5 times) for shorter times (<5 minutes), or when individual spikes (increase higher than 1 μS and back to previous level in 2 seconds or less) or drops (decrease of more than 1 μS and back to previous level in 2 seconds or less) occurred in the signal, as these may be considered as indication of loss of contact between the sensor and the skin.

Pre-Processing

In some embodiments, curation of training and evaluation data sets may include data analysis. Raw EDA data may be lowpass filtered (e.g., Butterworth filter of 4^{th} order; cutoff frequency of 0.4 Hz), smoothed (e.g., factor 9) and had spikes eliminated using a suitable filter function such as, e.g., medfilt1 function. Data start may be determined based on data quality and rounded up to the nearest complete hour. Data end may be rounded down to the previous complete hour. In the time domain, EDA level may be analyzed. In some embodiments, in the frequency domain, a wavelet may be calculated with a suitable wavelet function, such as, e.g., by the cwt function in MATLAB with Morlet template or with any other suitable tool set. In some embodiments, the power in the frequency range, e.g., of 0.01 to 0.3 Hz may be determined. While the EDA level may be affected by a variety of influencing factors, activity in low frequencies (such as up to 0.25 Hz) may be shown to be responsive to sympathetic tone arousal with comparably low intra-individual variability. As EDA relates to thermoregulation, surface body temperature recorded at the wrist or ankle (TEMP) by the same device may be analyzed. Raw TEMP data may be lowpass filtered (e.g., Butterworth filter of 4^{th} order; cutoff frequency of 0.4 Hz), smoothed (e.g., factor 9) and the level may be analyzed.

Data Analysis and Statistics

In some embodiments, further analyses may be performed to develop input features for the training and validation data sets. The analyses may include three different steps. For analysis steps 1 and 2, mean values of EDA level, EDA power, and TEMP level may be analyzed over all consecutive 60-minute windows of the entire recording. For analysis step 3, EDA signals starting 30 minutes prior to the seizure until 60 minutes after the seizure may be selected. Ictal data from the analysis may be excluded because signal quality may be often impaired due to movements, especially during convulsive seizures. First, the circadian EDA patterns in the entire data set across all patients may be described. The 24-hour pattern of (1) EDA level, (2) EDA power, and (3) temperature level may be modeled with nonlinear mixed-effects harmonic models and accounted for correlation between repeated measurements with a random intercept. Harmonic models refer to the statistical models in which the mean structure of the response variables may be represented by a Fourier series with a finite number of sine and cosine terms. Random effects may be imposed in such models to represent between-subject variation. Together, the random effects and representation of mean structure form nonlinear mixed-effects harmonic models for characterizing circadian rhythms. The number k denotes the number of sine and cosine functions (harmonic terms) and dominates the complexity of the models. Selection of an optimal k may be performed to balance on goodness-of-fit. In a first step, the model may be fit for the entire patient cohort with the number of harmonic terms ranging from 1 to 3 and then selected the best model based on the Bayesian information criterion (BIC). In some embodiments, the BIC, a criterion based in part upon likelihood for selection among a finite set of models, may be employed to select the optimal number k. Body temperature recorded at the wrist or ankle may be also analyzed to support the evaluation of potential confounding by skin temperature.

In some embodiments, differences in 24-hour circadian patterns of EDA recordings in patients with and without seizures may be examined. Therefore, the model may be fit for each group separately, again with 1, 2, or 3 harmonic terms to identify the best model based on BIC values. To compare the groups amongst each other, a nonlinear mixed-effects harmonic model that included group indicators and allowed each group to have a different circadian pattern may be fit. Then, likelihood ratio tests may be conducted between this complex model and the previous model that assumed a unified circadian pattern for all patients.

Thirdly, seizure-induced EDA responses may be described relative to the EDA level and power expected from the 24-hour modulation. The SZ group 24-hour modulated value at seizure time may be subtracted from the mean EDA level and power for the 30-minutes pre-ictal period (starting at 30 minutes prior to seizure onset and ending at the time of seizure onset), the 30-minutes post-ictal period (post-ictal I, 30 minutes after seizure offset) and the 60-minutes post-ictal period (post-ictal II, >30 to 60 minutes after seizure offset).

Generalized estimating equations (GEE) may be run with EDA level and EDA power as the dependent variables and seizure type (GTCS and FIAS, and other seizure types), patient ID as subject variable, as well as period (pre-ictal, post-ictal I, and post-ictal II; within-subject-variable), and the seizure type-by-time interaction as independent variables. The interaction terms characterized whether a change in outcome over time differed between the seizure types. To account for repeated seizures within the same patients, the autoregressive covariance structure may be chosen to adjust the estimator by number of patients and robust sandwich estimators may be obtained for variance estimation. In some embodiments, upon study, the statistical tests are two-tailed and have a significance level of p<0.05. in some embodiments, model effects may be expressed as mean percent change with 95% confidence intervals.

### Results

Patient Characteristics, Recordings, and Seizure Descriptions

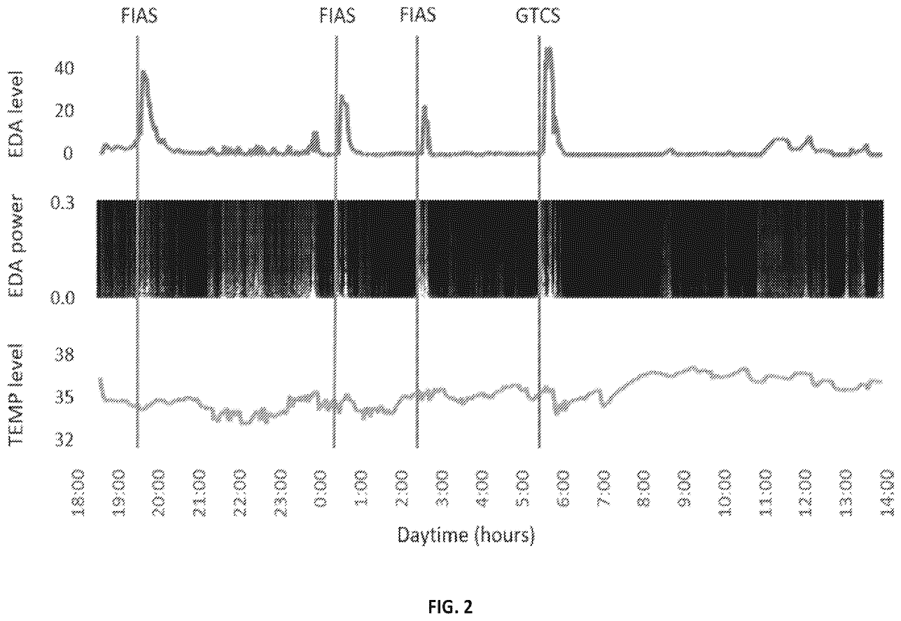

In some embodiments, to analyze 24-hour patterns, 2306 hours of recordings from a pediatric population of 119 patients may be included. The seizure group includes 40 patients (20 GTCS and 20 FIAS). The NEEG group have 17 patients, and the EA group includes 62 patients. Demographic and clinical characteristics are summarized for each subgroup in Table 1. The average length of recording per patient may be 19.4 hours (SD: 3.0 hours; min 8 hours and max 24 hours). After data cleaning, 59 seizures from 40 patients may be included. An example recording may be displayed in FIG. 2. Clinical and seizure characteristics based on seizure type are summarized in Table 2.

TABLE 1
Group-wise demographic and clinical characteristics of all patients
included in 24-hour EDA pattern analysis

| | | |
|---|---|---|
| | Epileptiform | |
| | activity without | |
| Normal EEG group | seizures group | Seizure group |
| (n = 17) | (n = 62) | (n = 40) |

Sex

| | | | | | | |
|---|---|---|---|---|---|---|
| Male | 11 | (65%) | 22 | (35%) | 25 | (62%) |
| Female | 6 | (35%) | 40 | (65%) | 15 | (38%) |

Age

| | | | | | | |
|---|---|---|---|---|---|---|
| In years, median (p25-p75) | 12.8 | (4.4-15.5) | 9.2 | (6.1-13.0) | 12.0 | (9.2-14.4) |

Epilepsy Diagnosis

| | | | | | | |
|---|---|---|---|---|---|---|
| Yes | 12 | (71%) | 52 | (84%) | 40 | (100%) |
| No/unknown | 5 | (29%) | 10 | (16%) | 0 | (0%) |

Etiology of epilepsy/seizure

| | | | | | | |
|---|---|---|---|---|---|---|
| Unknown | 6 | (35%) | 25 | (40%) | 14 | (35%) |
| Structural | 6 | (35%) | 22 | (36%) | 22 | (55%) |
| Genetic | 0 | (0%) | 5 | (8%) | 1 | (3%) |
| Metabolic | 0 | (0%) | 0 | (0%) | 0 | (0%) |
| Others | 0 | (0%) | 0 | (0%) | 3 | (7%) |
| Not applicable | 5 | (30%) | 10 | (16%) | 0 | (0%) |

Epileptiform activity on EEG

| | | | | | |
|---|---|---|---|---|---|
| Frequent and more than | N/A | 25 | (40%) | 14 | (35%) |
| focal | | | | | |
| Frequent and focal | N/A | 8 | (13%) | 5 | (12.5%) |
| Rare/occasional | N/A | 13 | (21%) | 5 | (12.5%) |
| and more than focal | | | | | |
| Rare/occasiona land | N/A | 16 | (26%) | 16 | (40%) |
| focal | | | | | |

Background on EEG

| | | | | | |
|---|---|---|---|---|---|
| Normal | N/A | 31 | (50%) | 13 | (33%) |
| Focal or intermittent | N/A | 17 | (27%) | 25 | (62%) |
| slowing | | | | | |
| Generalized and | N/A | 14 | (23%) | 2 | (5%) |
| continuous slowing | | | | | |

History of neurosurgery

| | | | | | | |
|---|---|---|---|---|---|---|
| Yes | 1 | (6%) | 4 | (6%) | 4 | (10%) |
| No | 16 | (94%) | 58 | (94%) | 36 | (90%) |

ASM during EMU stay

| | | | | | | |
|---|---|---|---|---|---|---|
| Levetiracetam | 5 | (29%) | 23 | (37%) | 19 | (48%) |
| Oxcarbazepine | 5 | (29%) | 15 | (24%) | 14 | (35%) |
| Carbamazepine | 0 | (0%) | 3 | (5%) | 2 | (5%) |
| Eslicarbazepine acetate | 0 | (0%) | 1 | (2%) | 1 | (2%) |
| Perampanel | 0 | (0%) | 1 | (2%) | 0 | (0%) |
| Clobazam | 3 | (18%) | 13 | (21%) | 15 | (37%) |
| Valproic Acid | 1 | (6%) | 9 | (15%) | 6 | (15%) |
| Lamotrigine | 1 | (6%) | 13 | (21%) | 5 | (13%) |
| Lacosamide | 2 | (12%) | 9 | (15%) | 12 | (30%) |
| Clonazepam | 2 | (12%) | 2 | (3%) | 5 | (12%) |
| Topiramate | 1 | (6%) | 3 | (5%) | 1 | (2%) |
| Vigabatrin | 0 | (0%) | 4 | (6%) | 0 | (0%) |
| Rufinamide | 0 | (0%) | 2 | (%) | 1 | (2%) |
| Zonisamide | 0 | (0%) | 5 | (8%) | 3 | (8%) |
| Phenobarbital | 0 | (0%) | 4 | (6%) | 2 | (5%) |
| Ethosuximide | 0 | (0%) | 3 | (5%) | 1 | (2%) |
| Fosphenytoin/phenytoin | 0 | (0%) | 4 | (6%) | 2 | (5%) |
| Cannabidiol | 0 | (0%) | 1 | (2%) | 2 | (5%) |
| Diazepam | 1 | (6%) | 1 | (2%) | 0 | (0%) |
| Gabapentin | 1 | (6%) | 2 | (3%) | 1 | (2%) |
| Lorazepam | 1 | (6%) | 7 | (11%) | 3 | (8%) |
| Midazolam | 0 | (0%) | 1 | (2%) | 0 | (0%) |
| Pyridoxine | 0 | (0%) | 3 | (5%) | 0 | (0%) |
| Oxazepam | 0 | (0%) | 0 | (0%) | 1 | (2%) |

Wristband location

| | | | | | | |
|---|---|---|---|---|---|---|
| Left wrist | 3 | (18%) | 14 | (23%) | 9 | (23%) |
| Left ankle | 5 | (29%) | 13 | (21%) | 7 | (18%) |
| Left unavailable | 1 | (6%) | 3 | (5%) | 0 | (0%) |
| Right wrist | 3 | (18%) | 18 | (29%) | 19 | (47%) |
| Right ankle | 3 | (18%) | 13 | (21%) | 5 | (12%) |
| Right Unavailable | 2 | (12%) | 1 | (2%) | 0 | (0%) |

MRI findings

| | | | | | | |
|---|---|---|---|---|---|---|
| Normal | 4 | (23%) | 22 | (35%) | 8 | (20%) |
| Abnormal | 10 | (59%) | 35 | (57%) | 32 | (32%) |
| Not done/not available | 3 | (18%) | 5 | (8%) | 0 | (0%) |

Legend.
ASM: anti-seizure medication.
EMU: epilepsy monitoring unit.
MRI: magnetic Where ASM: anti-seizure medication. EMU: epilepsy monitoring unit. MRI: magnetic resonance imaging. EEG: electroencephalogram.
p25-p75: percentiles 25th and 75th.
Descriptive data are presented as number of patients and percentages (n, %), except for age (in median, percentile 25, percentile 75).
*Percentages for ASM during EMU stay do not sum up 100% as many patients received more than one ASM.

TABLE 2
Clinical characteristics of patients included in the evaluation of seizure-
induced EDA responses

| | FIAS group | GTC group |
|---|---|---|
| Number of Seizures | 28 seizures, from 19 | 31 seizures, from 21 |
| and patients | patients | patients |
| Seizure duration in | 54 (48-91) [11-319] | 97 (80-111) [29-222] |
| seconds - median, | | |
| (percentile 25/75), | | |
| [range] | | |
| Seizure onset location | Temporal (L × 2) | Generalized (2) |
| | Frontal (L, R × 2) | Temporal (R × 6, L × 2) |
| | FT (L × 5, R × 4) | Frontal (L, R × 2) |
| | Bifrontal | Parietal (L × 4, R) |
| | Midline CP | FC (R) |
| | Anterior Q (R) | CT (R) |
| | Posterior Q (R × 2) | TP (R) |
| | Hemisphere (L × 2, | TO (L × 2) |
| | R × 7) | OP (R) |
| | | Bifrontal |
| | | Central (L, R × 2) |
| | | Parasagittal (L, R) |
| | | Posterior/parasagittal (R) |

Legend.
Pts: patients.
FT: fronto-temporal.
Q: quadrant.
TP: temporo-parietal.
TO: temporo-occipital.
OP: occipito-parietal.
FC: fronto-central.
CT: centro-temporal.
R: right.
L: left.
* For seizure onset location, we show the number of seizures in each location and the side within the parentheses. Examples: Temporal (L × 2): there are two seizures with left temporal onset. Bifrontal: there is one seizure with bifrontal onset, and side does not apply.

24-Hour Patterns of EDA Recordings in Patients with Epilepsy

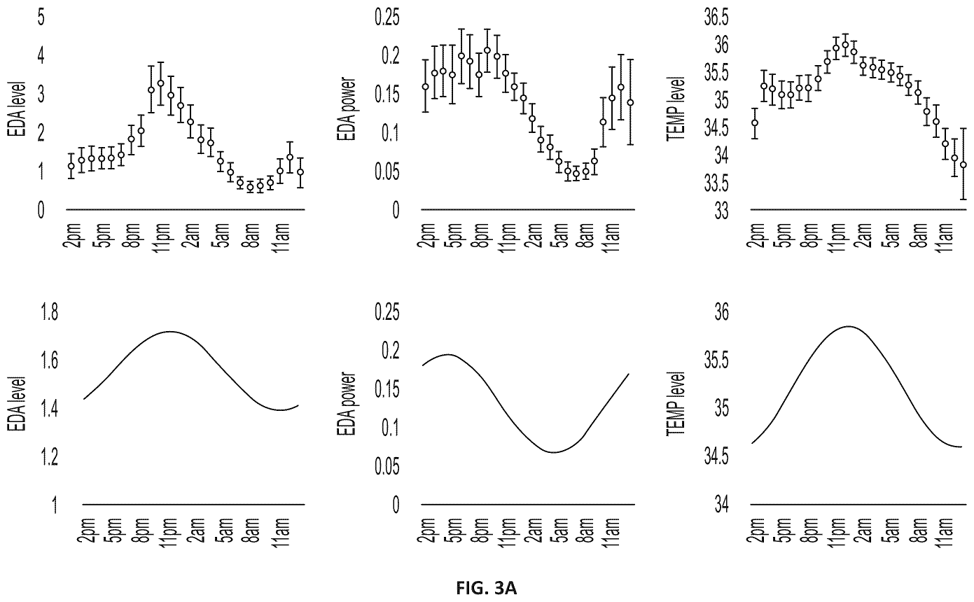

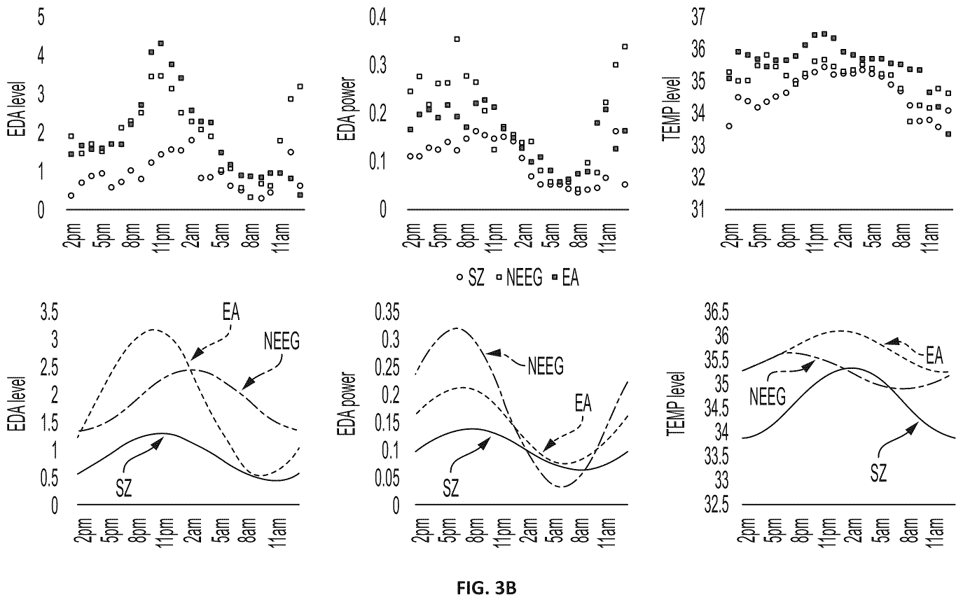

An example of measures resulting in mean and standard error values per hour of the day, excluding hours during which the seizure occurred, are presented in FIG. 3 for the overall patient group. In a first step, the optimal k for the nonlinear mixed-effects model may be tested for all patients together. Optimal k, indicated by lowest BIC for EDA level as well as EDA power, may be 2. For TEMP level, lowest BIC may be revealed for k=1, indicating a less complex sinusoidal pattern (Table 3 and FIG. 3). Maxima for EDA and TEMP levels may be located at night (EDA level at 11 p.m. and TEMP level 1 a.m.) and minima at 11 a.m. and 12 p.m. respectively. EDA power maximum may be located around 5 p.m. and the minimum around 5 a.m.

TABLE 3
Group-wise Bayesian information criteria (BIC) values for
EDA level, EDA power and temperature at wrist or ankle
BIC values

| | EDA level | | | EDA power | | | TEMP level | | |
|---|---|---|---|---|---|---|---|---|---|
| k | 1 | 2 | 3 | 1 | 2 | 3 | 1 | 2 | 3 |
| All | 1.08e+04 | 1.06e+04 | 1.06e+04 | −1.11e+03 | −999.73 | −749.33 | 8.44e+03 | 8.75e+03 | 8.77e+03 |
| SZ | 2.95e+03 | 2.96e+03 | 3.09e+03 | −633.97 | −632.32 | −470.13 | 3.08e+03 | 3.17e+03 | 3.24e+03 |
| NEEG | 1.60e+03 | 1.60e+03 | 1.63e+03 | 1.19 | 82.79 | 87.94 | 1.08e+03 | 1.11e+03 | 1.11e+03 |
| EA | 6.01e+03 | 5.85e+03 | 5.87e+03 | −535.93 | −550.70 | −362.96 | 4.11e+03 | 4.31e+03 | 4.14e+03 |

Legend.
BIC are presented for k = 1, 2, or 3 for the dependent variables electrodermal activity (EDA), EDA power, temperature (TEMP) for the entire research group (all) and per group, e.g., for seizure (SZ), normal EEG (NEEG), and epileptiform activity (EA) groups separately. Best model fit is highlighted.

Differences in 24-Hour Patterns of EDA Recordings in Patients with and without Seizures.

In some embodiments, BIC values for harmonic models per group are presented in Table 3 and illustrated in FIG. 3. For TEMP level, k=1 revealed the best fit for all groups. For EDA level, optimal k differed between groups, and for power, optimal k may be 2 for NEEG and EA groups and 1 for SZ group. For EDA power, models with k=1 fit best for SZ and NEEG groups, while k=2 revealed lowest BIC values for the EA group.

The comparison of H₀ (the small model not differentiating between groups), and H₁ (the larger model including terms per patient group), revealed that the model differentiating between groups (H₁) may be superior for EDA level (h=1; p<0.01) and EDA power (h=1; p<0.01), while H₀ represents data better for TEMP level (h=0; p=1).

In some embodiments, when H 1 may be superior, pairwise group comparisons may be continued. Follow-up analysis for EDA level and EDA power may compare restricted and unrestricted models per two groups. An example of the comparison of the EDA level pattern of the SZ group to the patterns of NEEG or EA groups, respectively, show that the groups differed (h=1; p<0.01), while patterns of NEEG and EA groups do not differ significantly (h=0; p=1). For an example of EDA power, all pairwise group comparisons reveal that the larger model differentiating between groups may be superior (h=1; p<0.01).

EDA Seizure Response Normalized to Respective 24-Hour Modulated SZ Group EDA Values

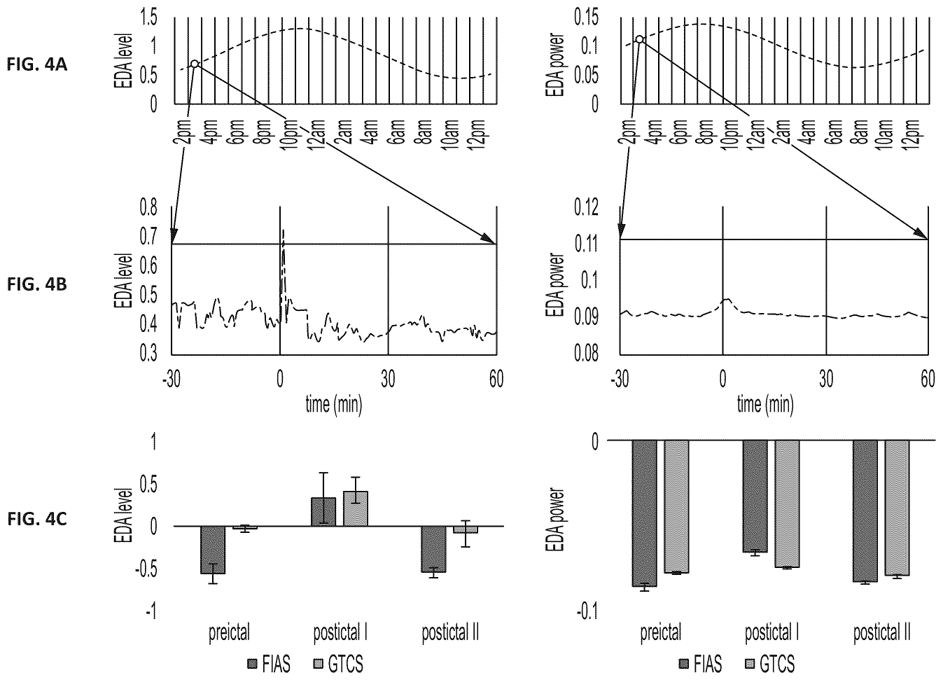

In some embodiments, to evaluate the seizure-induced changes in EDA level and power, the expected value may be subtracted based on the SZ group specific modeled 24-hour pattern at seizure time from the pre- and post-ictal periods (see FIG. 4). An example of results per seizure type are illustrated in FIG. 4. As shown in FIG. 4, EDA level of the pre-ictal period may be lower than expected, e.g., 40 out of 59 pre-ictal segments may be negative. During the post-ictal period I the values may be increased and then decreased in the post-ictal period II. EDA power may remain lower than expected for all segments, but showed an increase during the post-ictal period I.

In some embodiments, a GEE analysis having EDA level as outcome may be performed. In such a GEE analysis, a significant main effect period (χ²=16.40; p<0.001), and seizure type-by-period interaction (χ²=8.75; p=0.013) may be found. Main effect of seizure type may be not significant (χ²=0.11; p=0.739). An example of follow-up analysis of the interaction may reveal that GTCS and FIAS do not differ for the pre-ictal (p=0.814) or post-ictal period I (p=0.308). For post-ictal period II, GTCS EDA levels may be 8.44 times as high as FIAS' levels (CI: [1.47, 48.54]; p=0.017). For GTCS, EDA level of the post-ictal period I may be 11.93 times (CI: [2.61, 54.58]; p=0.001) and of the post-ictal period II may be 5.46 times (CI: [1.19, 25.00]; p=0.029) higher than levels of the pre-ictal period. For FIAS, EDA level of post-ictal I may be 30.23 times higher than the levels of the pre-ictal period (CI: [0.26, 2.97]; p=0.006).

In an example of EDA power, the main effect of seizure type (χ²=0.02; p=0.889) and the seizure type-by-period interaction (χ²=4.25; p=0.120) may be not significant, but the main effect of period (χ²=14.96; p=0.001) may be significant. An example of follow-up analysis may show that for the post-ictal period I, the EDA power may be higher, e.g., less negative, than the power during the post-ictal period II (1.01 times; CI: [1.01, 1.02]; p<0.001).

In some embodiments, taken together, results indicate that EDA response represents a relative and not an absolute increase, as pre-ictal values may be lower than the values expected from time-adjusted 24-hour modulation. The response may be higher and longer lasting for GTCS seizures than for FIAS. Accordingly, 24-hour patterns may be present in patients with epilepsy within EDA level and power with different peaks and troughs. Additionally, 24-hour patterns of EDA recordings differed between the groups with and without seizures, suggesting longer-term effects representing flattening before and after seizures. Pre-ictal periods exhibited lower values than expected based on 24-hour modeling of the SZ group's EDA pattern.

### Discussion

EDA May Represent an Important Measurement of Sympathetic ANS Activity.

In some embodiments, EDA may reflect the sympathetic nervous system through activity on the sweat glands and may be linked to arousal and other physiologic stimuli. The peri-ictal periods in patients with epilepsy, including focal and generalized seizures, exhibit signs of altered autonomic nervous system (ANS) function, commonly from its seizure-activated sympathetic component. EDA has been studied in patients with epilepsy as a measure of sympathetic activation; it may be shown that spontaneous epileptic seizures may be followed by an EDA elevation, also referred to as an EDA peak. As such, EDA activity may provide an approach to assess SUDEP risk and provide an important marker of dysautonomia associated central nervous system shutdown in SUDEP.

Circadian Patterns May be Reflected in EDA Recordings.

In some embodiments, the central regulation of circadian patterns plays a role in modulating the ANS, with the goal of maintaining homeostatic constancy, efficiency of physiological processes, and the adaptation to internal and external changes and requirements. The circadian timing system may be hierarchically organized and temporally controlled and coordinates multiple physiological systems with a central pacemaker in the suprachiasmatic nucleus. As ANS subsystems may be interconnected, they may be sensitive to control mechanisms within and across subsystems. Thermal changes may have a strong influence on the sweat gland activity. Therefore, temperature may be analyzed at wrist and/or ankle to show a simpler pattern, based on the optimal k=1. The pattern may be reflected in the EDA level and contribute to differences in timing of peaks of EDA level and EDA power. Accordingly, in some embodiments, EDA recordings may reflect more than thermoregulation, and that non-thermal factors might be related to central modulations. Power may be more sensitive to central autonomic arousal based on the peak in the evening.

Epilepsy Patients with and without Seizures During Video-EEG Presented with Different EDA Patterns.

In some embodiments, as described above, amplitudes of the 24-hour oscillation in EDA level and power may be lower in patients with seizures than in patients without seizures. In turn, there may be less variability in EDA patterns amongst patients with epileptic seizures, resulting in potentially reduced adaptability to internal and external stressors, such as seizures. Cardiac and respiratory ANS subsystems may be altered in people with epilepsy. In some embodiments, differences in patients with and without seizures may be detected on the recordings. Furthermore, differences between epilepsy patients during inter-ictal periods and healthy controls have been described in EDA responses with heterogeneous results showing either longer latencies or higher amplitudes. these findings may be aligned with marginally decreased EDA levels in patients with epilepsy compared to healthy controls. In some embodiments, within the group of epilepsy patients, the EDA level inversely correlated with seizure frequency in 10 minute recordings in relaxed state. Furthermore, modulation of the EDA level towards an increase may be related to reduced seizure frequency. This suggests potential bidirectional interaction of central and peripheral ANS parts and may open up further avenues for research and potential monitoring and intervention. Biological basis related to EDA regulatory effects and the changes throughout the daytime remain unclear, and suspected relationships, include enhanced functional connectivity between brain regions activated during executive control and attention allocation, also involving structures relevant for autonomic control.

In some embodiments, based on the bidirectional interactions of the circadian rhythm and the autonomic network, not only seizure occurrence likelihood, but also seizure induced alterations of the autonomic patterns may be relevant. One indicator for this may be the above-described flattening of the pattern that indicates more lasting seizure-related effects. The EDA level and power show reduced levels in pre-ictal periods for many patients irrespective of the time of occurrence in relation to the time-adjusted SZ group-specific 24-hour modulated values. In some embodiments, EDA may peak when in peri-ictal data, but as the increase may be relative to a level lower than normal, the peak may be not a prominent feature when reviewing entire recordings. This might reduce the specificity of EDA changes for seizure detection and suggests looking into the pattern of change in peri-ictal periods instead of comparing the periods as each has its own dynamical properties. The negative values may indicate a suppression of sympathetic activity prior to seizure onset, especially since the EDA power extracted for a frequency band may be most sensitive to central sympathetic control. these results may improve specificity of seizure detection by utilizing modulated values as a baseline and calculating relative changes and peaks.

EDA Response May be More Prominent in GTCS than FIAS.

In some embodiments, a sympathetic skin response in EDA level and power within the comparison of pre- and post-ictal periods may be higher and more long-lasting after GTCS than after FIAS. GTCS may therefore lead to greater perturbations of the ANS than FIAS.

In some embodiments long-term monitoring by the wearable sensor 101 may be performed in the inpatient and outpatient settings for predictive seizure risk alerts based on the correlation between EDA and pre-ictal periods. These recordings would allow for individualized monitoring aimed at detecting systematic seizure-induced changes. The reduced EDA on days with seizures and in pre-ictal periods may contribute to seizure detection and prediction, e.g., using the seizure model engine 120. The existence of a 24-hour pattern suggests including time of day in further detection and prediction approaches, so that EDA can be quantified according to 24-hour modulated values. The altered autonomic control of circadian rhythms, especially in GTCS, may also relate to SUDEP risk, as the reestablishment of the bidirectional interactions of peripheral and central structures after seizures seems crucial to regain homeostasis.

### CONCLUSION

Accordingly, in some embodiments, EDA recordings from wearable sensors 101 offer a non-invasive tool to continuously monitor sympathetic activity. In some embodiments, the exemplary data and analysis thereof as described above may be used as training and validation data for the seizure model engine 120 to learn the correlations between EDA and circadian cycles. In patients with epilepsy, EDA level and power may be affected by day and nighttime. EDA patterns differ from the TEMP pattern. Post-ictal EDA responses may be confirmed to last longer in GTCS compared to FIAS, and described a relationship between seizures and EDA patterns, as well as potential interactions between seizure patterns and 24-hour EDA patterns. Seizures occur in relation to EDA patterns, and even affect the pattern, e.g., resulting in lower amplitude and level of oscillation. Seizure-related responses start from reduced EDA levels in the pre-ictal period and evolve into relatively high EDA responses in post-ictal periods. In some embodiments, these seizure-related changes surrounding the possible rhythmic nature of EDA provide a relevant biomarker for seizure detection and prediction by the trained seizure model engine 120.

### Example Embodiment 2

### Materials and Methods

In some embodiments, in a first example embodiments, patients may be provided with the wearable sensor 101 such as a wearable biosensor including, e.g., a wrist- or ankle-worn sensor such as, e.g., an E4 biosensor, an Apple Watch, a Samsung Galaxy Watch, a FitBit, or other suitable smartwatch, health tracker or biometric tracking wearable or any combination thereof. In some embodiment, the wearable sensor 101 may provide ANS measurements to a seizure monitoring system 110 to use machine learning to predict whether a coming period is of high risk or low risk for a seizure. In some embodiments, the patients may include patients who have at least one generalized tonic-clonic seizure (GTCS) or focal impaired awareness seizure (FIAS) during video-EEG while wearing the wearable sensor 101 (SZ), or who do not have seizures during video-EEG (no-SZ). In some embodiments, patients with status epilepticus (seizures longer than 10 minutes for FIAS and 5 minutes for GTCS) may be excluded. In some embodiments, if multiple recordings are available per patient, the earliest recording may be included, favoring the right body side to equalize the number of recordings of the left and right sides of the body, as the sensor was placed more often on the left body side in this dataset. In some embodiments, the sensor location does not differ between groups (see Table 5). For the SZ group, the recording that include at least one seizure may be selected. If multiple recordings contained seizures, the one with fewer seizures may be selected to maximize inter-ictal recording length.

Data Recording and Quality Check.

In some embodiments, the wearable sensor 101 may capture electrodermal activity (EDA, sampling rate 4 Hz), peripheral body temperature (TEMP, sampling rate 4 Hz), and heart rate (HR, sampling rate 1 Hz). The recordings started between 9 a.m. and 4 p.m. To allow wristbands to calibrate and to exclude the wristband removal time from the recording, we excluded the first 30 minutes and last 10 minutes of each recording. The recording start time may be rounded up to the nearest 10-minute increment (e.g., a recording start time of 9:47 a.m. was rounded to 9:50 a.m.). In some embodiments, a data quality check may be performed per 10-minute segment. In some embodiments, a quality check fails when 10-minute mean values has either an EDA level lower than 0.05 μS, a TEMP lower than 20° C. or higher than 40° C., or an HR lower than 45 bpm or higher than 200 bpm or any combination thereof. If the data quality check fails, the associated segments may be excluded from the analysis. For SZ patients, three 10-minute pre-ictal segments and six post-ictal 10-minute segments may be excluded, including a segment during which a seizure occurred. After the quality check and seizure time exclusion, any remaining patients with less than clean segments may be excluded to ensure that 24-hour modeling is based on a recording length over 13 hours. Some patients may be enrolled on multiple days during the same EMU stay. For the above-mentioned analysis, one recording per patient may be employed. If there are multiple recordings for a patient, the recording with a seizure may be selected and between multiple recordings with a seizure, the first recording in time may be selected during the admission for the main analysis. To obtain insights into within-patient effects, seizure-free recordings may be analyzed for the SZ patients, when available. Seizure-free recordings may be recorded one or two days before the seizure recording and 24-hour patterns may be modeled and amplitude and level for EDA, HR, and TEMP may be calculated as described above.

Clinical Data Collection.

In some embodiments, clinical data may be collected for patients that pass the data quality check. Using clinical notes, age, sex, age of first seizure, etiology of epilepsy, MRI findings, seizure frequency, reduction in anti-seizure medications (ASM) during the hospital stay, interictal abnormalities, (e.g., normal EEG, spikes, focal slowing, generalized slowing) among other clinical data or any combination thereof may be collected. In some embodiments, where seizure frequency values are missing the seizure frequency may be set as the group mean to include patients for the overall analysis. In some embodiments, per ILAE 2017 guidelines, a board-certified epileptologist may review the video-EEG recordings to determine seizure type and electrographic seizure onset and offset times. Tonic-clonic seizures of focal and generalized onset as GTCS may also be classified.

Data Analysis and Statistics.

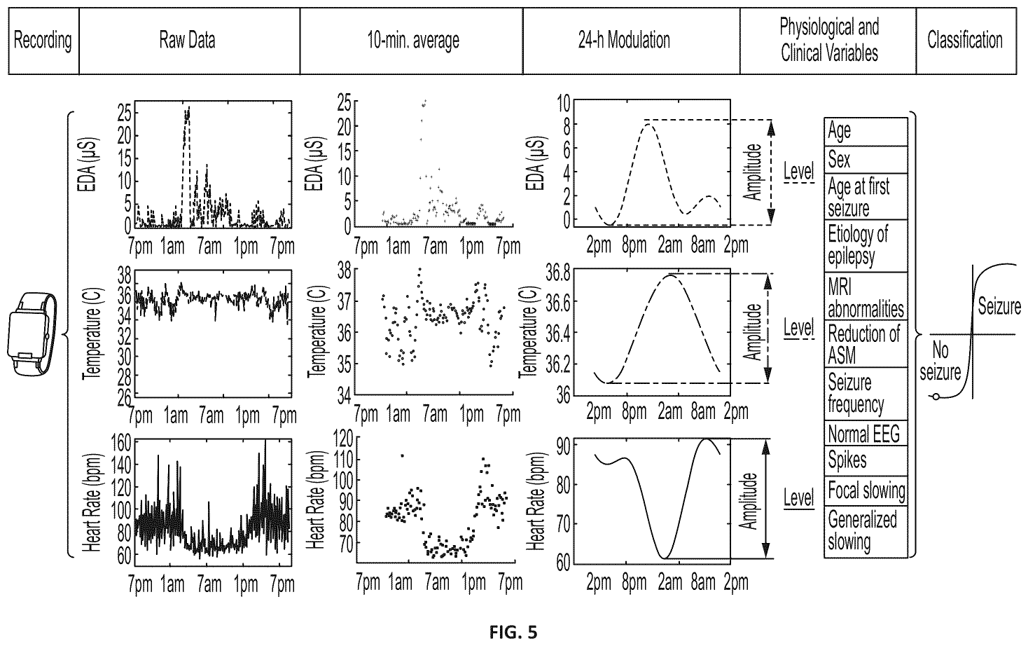

FIG. 5 depicts a schematic illustration of data collection and analysis steps in a second example embodiment according to aspects of the present disclosure. The schematic illustration includes (from left to right) recording with the wearable wristband, raw data processing, averaging of data over 10-min-segments, 24-hour pattern modulation modeling (cycle start: 2 pm), amplitude and level calculation, adding clinical variables, and classification into a seizure or a non-seizure recording.

In some embodiments, data analysis may be performed using a suitable toolkit such as, e.g., MATLAB or other suitable data analysis software. In some embodiments, EDA, TEMP, and HR recorded values may be averaged over segments of a predetermined length, such as, e.g., 10-minute segments, for data analysis. In some embodiments, a nonlinear mixed-effects harmonic model may be employed to model the 24-hour pattern of EDA, TEMP, and HR and calculated the modulation's mean level and amplitude from the resulting curve of each patient. In some embodiments, the modelling may result in two-tailed statistical tests and a significance level may be predetermined at a suitable significance level, such as, e.g., 0.05. In some embodiments, univariate logistic regression may be performed to test for group differences in modulation level and amplitude of EDA, TEMP, and HR.

In some embodiments, for variable selection, a multivariate logistic regression with a stepwise backward selection of physiological and clinical variables by Bayesian Information Criteria may be performed. The variables selected by the stepwise backward selection in multivariate logistic regression may be included for further supervised learning. In some embodiments, one or more supervised learning algorithms may be implemented. In some embodiments, the performance of the following five learning algorithms along with logistic regression may be assessed: K-nearest neighbor, random forest, Ada Boost, Gaussian naive Bayes, and support vector machine (SV; linear and nonlinear with Radial Basis Function (RBF) kernel). In some embodiments, to assess each algorithm, cross-validation may be employed, such as, e.g., 10-fold cross-validation. Thus, in some embodiments, cross-validation may be performed including randomly shuffling the data labels 10 times and statistically compare the performance of each machine learning algorithm for the shuffled labels to the original labels, e.g., by t-test. For LR, KNN, RF, AB, and GNB classifiers, the prediction performance may be validated by Brier score. For within-patient comparison, repeated-measure ANOVA may be performed, with recording time (seizure-free recording before seizure recording and seizure recording) as the repeated factor.

### Results

Patient Characteristics, Recordings, and Seizure Descriptions.

In some embodiments, for an exemplary 117 patients, forty-nine patients may form the seizure group (8 patients with FIAS, 26 patients with GTCS, and 15 patients with both FIAS and GTCS), and 67 patients may form the no-seizure group (see, FIG. 5). Example demographic and clinical characteristics are summarized in Table 4 for each subgroup. Additional example seizure information is presented in Table 5 below for the seizure group.

TABLE 4
Group-wise demographic and clinical characteristics of all patients included in an
example 24-hour EDA pattern analysis. Chi-square tests may be calculated for
categorical and Mann-Whitney-U tests for continuous variables to compare groups.

| | No-Seizure | | Seizure Group | | |
|---|---|---|---|---|---|
| | Group (n = 67) | | (n = 49) | | p |
| Sex | | | | | 0.098 |
| Male | 30 | (44.1%) | 30 | (61.2%) | |
| Female | 39 | (55.9%) | 19 | (38.8%) | |
| Age at Enrollment | | | | | 0.019 |
| In years, median (IQR, p25- p75) | 9.4 | (8.4, 7.0-15.4) | 13.2 | (7.2, 9.6-16.8) | |
| Age at First Seizure | | | | | 0.021 |
| In years, median (IQR, p25- p75) | 3.5 | (5.9, 1.1-7.0) | 7.0 | (8.5, 2.0-10.5) | |
| Seizure Frequency (estimated per 30 days) | | | | | 0.839 |
| No per month (IQR, p25-p75) | 4.0 | (20.3, 0.7-20.9) | 4.0 | (9.7, 1.0-10.7) | |
| Etiology of Epilepsy | | | | | 0.219 |
| Structural | 22 | (32%) | 23 | (47%) | |
| Unknown | 36 | (53%) | 20 | (41%) | |
| Genetic | 6 | (12%) | 3 | (6%) | |
| Immune | 1 | (1%) | 2 | (4%) | |
| Infectious | 0 | (0%) | 1 | (2%) | |
| Metabolic | 0 | (0%) | 0 | (0%) | |
| Not Reported | 3 | (4%) | 0 | (0%) | |
| Interictal EEG | | | | | |
| Normal | 13 | (19%) | 4 | (8%) | 0.097 |
| Spikes | 55 | (81%) | 43 | (88%) | 0.320 |
| Focal Slowing | 14 | (24%) | 23 | (47%) | 0.002 |
| Generalized Slowing | 10 | (17%) | 0 | (0%) | 0.005 |
| MRI Findings | | | | | 0.050 |
| Normal | 20 | (29%) | 6 | (12%) | |
| Abnormal | 33 | (49%) | 25 | (51%) | |
| Not done/not available | 15 | (22%) | 18 | (37%) | |
| Reduction of at least 1 ASM During EMU Stay | | | | | 0.001 |
| Yes | 18 | (26%) | 31 | (63%) | |
| No | 46 | (68%) | 18 | (37%) | |
| Not available | 4 | (1%) | 0 | (0%) | |
| Wristband Location** | | | | | 0.350 |
| Left wrist | 12 | | 13 | | |
| Left ankle | 12 | | 18 | | |
| Left unavailable | 3 | | 0 | | |
| Right wrist | 17 | | 13 | | |
| Right ankle | 9 | | 4 | | |
| Right unavailable | 1 | | 0 | | |
| Unavailable | 0 | | 1 | | |
| Recording length in hours (after 10 min segment exclusion) | | | | | 0.014 |
| Mean (p25-p75) | 20.94 | (18.5-23.0) | 19.33 | (17.0-21.3) | |

TABLE 5
Seizure Characterization
Number of seizures during analyzed recordings (patient n = 49;
seizure n = 137)

| | | |
|---|---|---|
| Mean (min-max) | 2.8 | (1-11) |
| Seizure Type, patient n (seizure n) | | |
| GTCS | 26 | (62) |
| FIAS | 8 | (21) |
| Both | 15 | (54) |
| Seizure Timing, seizure n | | |
| 2:00 pm to 7:59 pm | 28 | |
| 8:00 pm to 1:59 am | 32 | |
| 2:00 am to 7:59 am | 38 | |
| 8:00 am to 1:59 pm | 39 | |
| Vigilance State, seizure n (%) | | |
| Sleep | 51 | (37.2) |
| Awake | 79 | (57.7) |
| Not Available | 7 | (5) |
| Seizure Duration, seconds | | |
| Mean (min-max) | 70.7 | (2-1378) |
| Seizure Localization and Lateralization by most | | |
| frequent onset zone**, seizure n | | |
| Generalized | 31 | |

| | |
|---|---|
| Temporal | L: 39, R: 16 |
| Frontal | L: 6, R: 9, B: 7 |
| Parietal | L: 7, R: 4, M: 2 |
| Parasagittal | R: 3 |
| Posterior | R: 2 |
| Central | L: 8, R: 5, M: 6 |
| Occipital | L: 4 |
| Hemisphere | L: 4, R: 8 |
| Unknown/Not Available | M: 2 |

Abbreviations:
GTCS, generalized tonic-clonic seizures;
FIAS, Focal impaired awareness seizures;
R, right;
L, left;
B, bilateral;
M, midline.
*Seizure duration is calculated for isolated seizures only, not clusters of seizures. The dataset contains 3 total clusters from 2 patients.
**For seizure-onset localization, the number of seizures in each location and the lateralization are reported. Seizures can occur in multiple locations. Examples: Temporal (L: 2): There are two seizures with left temporal onset. Generalized (1): there is one seizure with generalized onset, and the lateralization does not apply.

Modulation Pattern Differences.

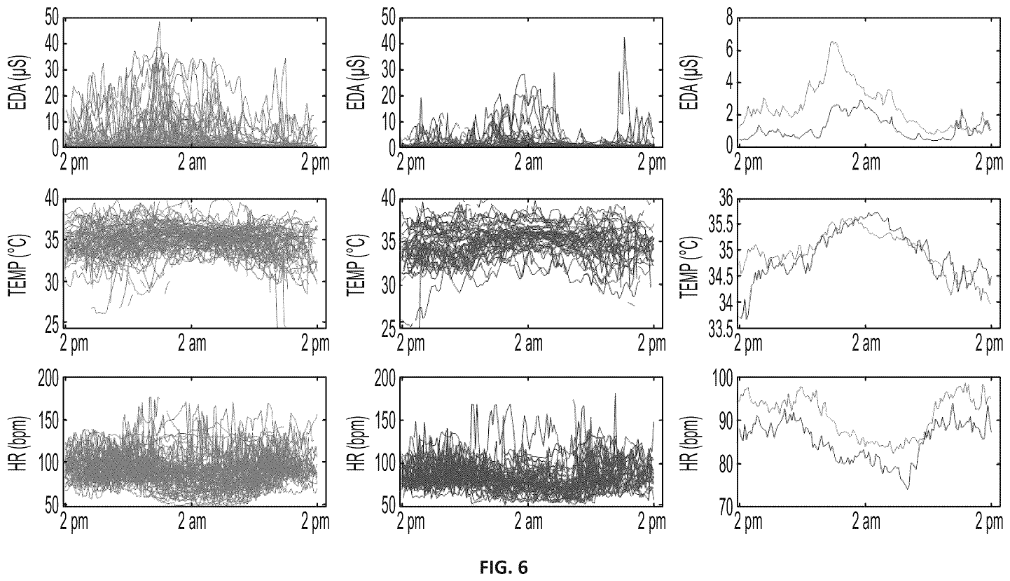

FIG. 6 depicts individual recordings of EDA, TEMP, HR (from top to bottom) averaged over 10-min segments of no-seizure (left panel) and seizure patients (middle panel) are displayed over 24 hours in a second example embodiment according to aspects of the present disclosure. The right panel shows the mean curves of respective autonomic modalities for no-seizure and seizure patient groups.

Of the example 117 patients, individual recordings may reveal a pattern of change over time for both groups and all modalities (FIG. 6). While EDA and TEMP peak at night, HR may decrease during the night and show a trough in the morning hours, starting around, e.g., 6 AM. TEMP may be peripherally recorded at the wrist and/or ankle and therefore may vary from core temperature curves. EDA level and amplitude, as well as tendencies for HR level, may show group differences with lower values for the seizure group. Descriptive group statistics and univariate logistic regression p-values for modulation level and amplitudes of EDA, TEMP, and HR are summarized in Table 6 below.

TABLE 6
Group-wise summary of modulation level and amplitude of the example 24-hour modulation of EDA, TEMP,
and HR. Mean, standard deviation (SD), median, and 25th and 75th percentiles are presented for the no-
seizure and seizure groups. P = values of univariate logistic regressions are presented in statistics.

| No-seizure group | | | | | Seizure group | | | | | |
|---|---|---|---|---|---|---|---|---|---|---|
| | | | Percentile | Percentile | | | | Percentile | Percentile | Statistics |
| Mean | SD | Median | 25 | 75 | Mean | SD | Median | 25 | 75 | p-value |

Level of 24-hour modulation

| | | | | | | | | | | | |
|---|---|---|---|---|---|---|---|---|---|---|---|
| EDA (μS) | 2.31 | 3.38 | 1.21 | 0.30 | 2.58 | 1.11 | 1.17 | 0.74 | 0.34 | 1.29 | 0.031 |
| TEMP (° C.) | 35..02 | 1.29 | 35.02 | 34.02 | 35.90 | 34.96 | 1.35 | 34.73 | 34.34 | 35.84 | 0.804 |
| HR (bpm) | 91.05 | 14.16 | 91.11 | 79.70 | 99.89 | 86.17 | 12.52 | 83.92 | 77.81 | 93.97 | 0.060 |

Amplitude of 24-hour modulation

| | | | | | | | | | | | |
|---|---|---|---|---|---|---|---|---|---|---|---|
| EDA (μS) | 5.33 | 6.20 | 3.26 | 0.45 | 8.14 | 3.18 | 4.21 | 1.41 | 0.59 | 4.14 | 0.045 |
| TEMP (° C.) | 1.67 | 1.17 | 1.46 | 0.82 | 2.29 | 2.05 | 1.62 | 1.54 | 0.99 | 2.66 | 0.148 |
| HR (bpm) | 224.28 | 10.52 | 22.31 | 16.39 | 31.25 | 24.04 | 10.91 | 22.72 | 15.03 | 31.52 | 0.906 |

Predictor Selection.

In some embodiments, according to the example 117 patients and the above described training and validation, the best model (AIC=110.8, BIC=141.2) may include EDA level (p=0.015), sex (p=0.011), age at first seizure (p=0.036), MRI findings (p=0.001), reduction of ASM (p<0.001), normal EEG (p<0.001), spikes (p=0.011), and generalized slowing (p<0.001). This model may have an accuracy of 83.8%, a sensitivity of 88.2%, a specificity of 77.6%, and an AUC-ROC of 0.907 (CI: 0.854-0.960).

Differentiating Recordings with and without Seizures.

In some embodiments, on average, cross-validated machine learning models differentiate between groups with an accuracy of 0.71 and AUC-ROC of 0.77 (see Table 7 below for example individual classifier performance). In some embodiments, label shuffling reveals that classification results may differ from chance (mean accuracy of 10 shuffles=0.55; p<0.001 for all classifiers). The average Brier score is 0.21.

TABLE 7
Classifier performance for the distinction between
recordings of patients with and without seizures.

| | | | | Brier | AUC | Shuffle | |
|---|---|---|---|---|---|---|---|
| Classifier | Accuracy | Sensitivity | Specificity | Score | ROC | Accuracy (SD) | P-value |
| Logistic Regression | 0.74 | 0.69 | 0.7800 | 0.19 | 0.81 | 0.53 (0.047) | <0.001 |
| K-Nearest Neighbors* | 0.74 | 0.53 | 0.9000 | 0.20 | 0.75 | 0.57 (0.032) | <0.001 |
| Random Forest | 0.69 | 0.55 | 0.7800 | 0.25 | 0.71 | 0.51 (0.058) | <0.001 |
| Ada Boost* | 0.68 | 0.65 | 0.6900 | 0.22 | 0.74 | 0.52 (0.048) | <0.001 |
| Naive Bayes* | 0.69 | 0.49 | 0.8400 | 0.23 | 0.77 | 0.58 (0.008) | <0.001 |
| Linear SVM | 0.73 | 0.63 | 0.7900 | 0.19 | 0.80 | 0.56 (0.023) | <0.001 |
| RBF SVM | 0.69 | 0.53 | 0.8100 | 0.19 | 0.78 | 0.56 (0.042) | <0.001 |

The area under the curve for receiver operating characteristics (AUC ROC); Mean 0 = mean of predicted values for patients of the without seizure group; Mean 1 = mean of predicted values for patients of the with seizure group;
*Parameters may be tuned: K-Nearest Neighbors (N from 2 to 10), Ada Boost (learning rate: 0.2, 0.1, 0.09, 0.08, 0.07, 0.06, 0.05, 0.01), naïve Bayes (var-smoothing: 2 to 6).

Within Patient Comparison of EDA, TEMP, and HR Levels and Amplitudes.

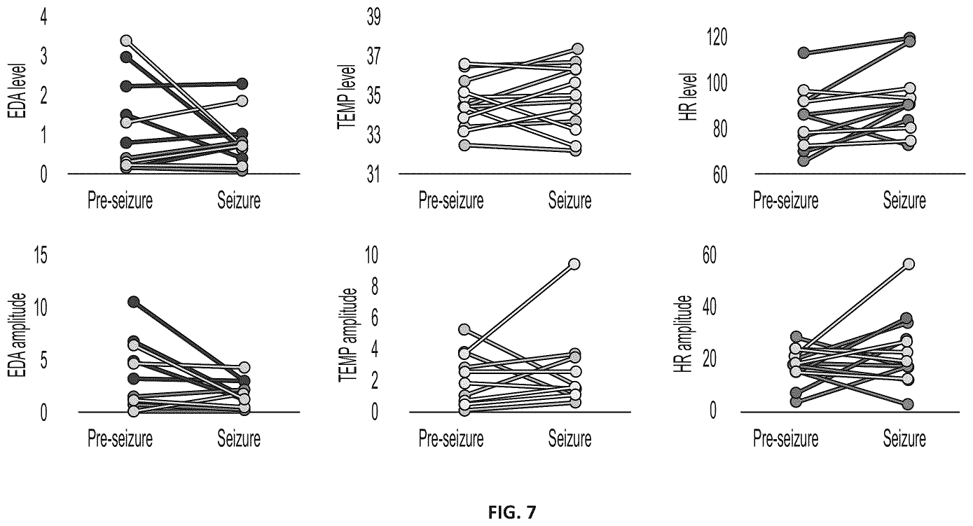

FIG. 7 depicts an example within patient comparison of ANS measurements in the second example embodiment according to aspects of the present disclosure.

In some embodiments, HR levels may be lower in pre-seizure compared to seizure recordings (F(1,13)=6.68, p=0.02, ηₚ²=0.34). EDA amplitudes (F(1,13)=3.46, p=0.09, ηₚ²=0.21) may trend lower in pre-seizure compared to seizure recordings. Modulation TEMP levels and amplitudes (level: F(1,13)=0.94, p=0.35, ηₚ²=0.07; amplitude: F(1,13)=0.81, p=0.38, ηₚ²=0.06), HR amplitude (F(1,13)=2.01, p=0.18, ηₚ²=0.13) and EDA level (F(1,13)=1.27, p=0.28, ηₚ²=0.09) may not differ between recordings.

### Discussion

In some embodiments, patient-specific analysis and the inclusion of clinical variables and HR recordings may lead to improved differentiation between patients who had a seizure or not during a recording. Patient-level analysis may be used to test characteristics of the 24-hour pattern as a biomarker for seizure monitoring. In some embodiments, following a multimodal approach, characteristics of 24-hour patterns of HR, EDA, and TEMP may be analyzed to classify patients into those with and without seizures. In some embodiments, such analysis shows that patients with seizures have lower EDA levels and amplitude and lower HR as compared to patients without seizures. In some embodiments, feature selection may reveal that combining EDA mean levels with clinical variables produces the best model and differentiates better than chance between patient groups. Comparing pre-seizure to seizure recordings within the same patient suggests that changes happen before the seizure day and, consequently, physiological markers may be predictive. Accordingly, the physiological markers, such as EDA, HR, TEMP, or other ANS measurements may be used to train and implement a machine learning algorithm, e.g., via the seizure model engine 120 to classify a risk of seizure.

In some embodiments, 24-hour patterns of peripherally recorded autonomic activity differ between recordings with and without seizures within and across participants. Central regulations of circadian patterns modulate ANS activity. As a result, ANS subsystem activity shows interconnected 24-hour patterns that change based on the disease state. For epilepsy patients, 24-hour patterns may be affected by long- and short-term alterations of autonomic functioning. While individual seizures manifest in acute autonomic responses, recurrent seizures and changes in central structures may cause long-term changes in autonomic control and regulations. Heart rate, body temperature, and EDA exhibit circadian variation. The suprachiasmatic nucleus, responsible for the body's circadian control, guides the autonomic outputs to maintain homeostasis and the organized physiological shifts between sleep and awake. Disruption of this control may increase susceptibility to disease. In healthy subjects, HR peaks early in the afternoon and drops during the night. Sweating threshold and skin temperature are highest in the evening and lowest past midnight. Conversely, EDA peaks past midnight and is lowest during late afternoon hours, which is similar to the no-seizure group results. In some embodiments, upon finding that 24-hour modulation patterns in EDA recordings show a seizure-related lower amplitude and level of the curve, a multimodal analysis reveals a lower HR level in the 24-hour modulations, confirming the effects of epilepsy on cyclic regulation of the cardiorespiratory system.

Furthermore, in some embodiments, the patient-specific analysis may show that HR levels are lower while EDA amplitudes tend to be higher in pre-seizure compared to seizure recordings. This result suggests HR is altered before a seizure and is a step towards understanding the 24-hour modulation curve flattening as a pre- or post-ictal phenomenon and that seizure-related autonomic changes might occur on different time scales for different modalities, e.g., following a multimodal pattern.

The Combination of Physiological and Clinical Variables to Distinguish Between Recordings with and without Seizures.

In some embodiments, the feature selection process may utilize the unique properties of EDA responses to seizures. Example results indicate that combining 24-hour EDA level with select clinical variables classifies best between patient groups. Thus, peri-ictal changes to induce a pattern of change that is constant across patients may be predicted. However, while some similarities exist across patients, ANS activity may vary between and within individuals.

In some embodiments, individual clinical characteristics affect ANS modulation in patients with epilepsy. By including clinical variables, variability across patients may be accounted for. In some embodiments, the final classification model may include input features including sex, epilepsy diagnosis, age at first seizure, MRI findings, reduction of ASM during the hospital stay, normal EEG, spikes, and generalized slowing. Females generally may have higher parasympathetic activity, whereas males may have higher sympathetic surges. The age at first seizure may relate to developmental processes and indicates the duration of epilepsy as well, which may result in the manifestation of seizures over time. Furthermore, structural brain abnormalities seen on MRI may alter or disrupt the pathways and processes of the central autonomic network. The reduction of ASM is meant to induce seizures during the stay and EEG and ANS monitoring, and analysis results may confirm the efficacy of it. However, it might be that the group of patients with no reduction consists of two types of patients, one that seizes without reduction and one that does not have a seizure during the stay. Furthermore, interictal EEG findings may contribute to the classification of patients with and without seizures, for example, 22 patients may have a normal EEG and may be diagnosed with epilepsy, while more of those patients may be in the no-SZ than in the SZ group. In some embodiments, two types of epileptiform activity, e.g., spikes and general slowing may contribute to the predictive model. Other suitable biometric and clinical data may be included in any suitable combination.

Monitoring 24-Hour Modulation Patterns in the Context of Seizure Detection and Prediction.

In some embodiments, the seizure model engine 120 may implement a machine learning algorithm as described above to distinguish between recordings with and without seizures based on machine learning-based classifications. In some embodiments, pre-ictal or post-ictal, or both may effect 24-hour modulations. Thus, the pre-ictal and/or post-ictal effects along with ANS measurements and/or clinical data may be used as biomarkers for seizure detection, prediction, and forecasting using a suitable machine learning algorithm. In some embodiments, group-based monitoring approach may be combined with patient-specific approaches presented for seizure prediction and detection. Accordingly, in some embodiments, estimating seizure risk for a coming period based on group classification may be performed after one day of recording and without the occurrence of a seizure. Thus, training the machine learning model as described above may enable patient-specific approaches to individualized seizure monitoring, particularly where recordings with seizures are available for a particular patient.

In some embodiments, the 24-hour pattern biomarkers can be further combined with existing forecasting approaches. Besides physiological data, seizure diaries and spike evaluations from EEG recordings have been successfully tested as seizure forecasting tools. In the outpatient setting, seizure diary data may be used to monitor seizures. In some embodiments, in the inpatient setting, specifically during video-EEG monitoring, spikes may be used to train seizure forecasting models against the wearable sensor data 102. Both approaches involve cyclic seizure patterns and may be related to the 24-hour patterns.

### CONCLUSION

Seizure-induced changes of autonomic activity affect 24-hour modulation patterns in individuals. Differences are characterized by lower activity and smaller deflections on a 24-hour scale. Within-patient comparison shows that ANS changes occur before seizures with different timing for different modalities. Moreover, monitoring epileptic seizures based on changes in 24-hour EDA patterns from wearable recordings (e.g., the wearable sensor data 102) may be performed, e.g., alone, or when combined with clinical variables. Accordingly, training a machine learning algorithm to classify a current and/or future period in a 24-hour cycle based on training against video and/or EEG monitoring-based ground-truth data.

### Example Embodiment 3

In some embodiments, short ANS recordings from the wearable sensor 101 may be used as an input to a machine learning algorithm for potential seizure likelihood assessment. In some embodiments, the short ANS recording may include, e.g., 15-minute ANS recordings in an evening period (e.g., 9:00 to 9:15 p.m. or other suitable evening period prior to entering a sleeping stage of a circadian cycle) and a morning period (e.g., 6:00 to 6:15 a.m. or other suitable morning period upon entering a waking period of the circadian cycle). ANS activity may differ between patients with and without impending seizures, and that group differentiation may be achieved by physiological variables from short-term recordings with or without additional clinical data.

### Methods

In some embodiments, the wearable sensor 101 may be positioned, e.g., on a wrist and/or ankle during video-EEG to generate training and/or validation data sets for producing a trained machine learning algorithm for use by the seizure model engine 120 to detect and/or predict seizure likelihood based on wearable sensor data 102. In some embodiments, the training and/or validation data may include patients with no seizures, at least one generalized tonic-clonic seizure (GTCS), and/or at least one focal impaired awareness seizure (FIAS). In some embodiments, the training and/or validation data may exclude patients with status epilepticus (seizures longer than 5 or 10 minutes for GTCS or FIAS, respectively) and/or with only other seizure types during the recording. In some embodiments, where multiple recordings are available for a given patient, the earliest recording with seizures may be included. Where recordings from both body sides are available, the left may be selected over the right body side, assuming to have mostly right-handers and consequently signal quality to be higher for the non-dominant hand.

Data Recording and Processing.

In some embodiments, to curate the wearable sensor data 102, recordings from patients may be include the following signals: EDA at a suitable sampling rate (e.g., 4 Hz), peripheral body temperature (TEMP) at a suitable sampling rate (e.g., 4 Hz), and heart rate (HR) at a suitable sampling rate (e.g., 1 Hz) among other ANS measurement recordings. In some embodiments, the root mean square of successive differences (RMSSD) may be calculated from the inter-beat interval (IBI). In some embodiments, the training and/or validation data sets may be pruned to include two sets of short recordings in the evening (e.g., from 9:00 to 9:15 p.m.) and in the morning (e.g., from 6:00 to 6:15 a.m.). In some embodiments, the recordings may be screened and/or filtered to ensure that TEMP is above 20° C. or below 40° C. on average, EDA is above 0.05 μS, and HR is between 45 and 200 beats per minute.

In some embodiments, the clinical data may be added to the wearable sensor data 102. In some embodiments, the clinical data may include, e.g., chart review of clinical notes, e.g., according to a standardized clinical data acquisition tool. In some embodiments, the following variables may be collected and stored: sex, age during enrollment, age at first seizure, MRI finding, anti-seizure medication (ASM) reduction during stay, generalized slowing on interictal EEG, interictal spikes, and seizure before measurement during the same recording. In some embodiments, to create the training and/or validation data, a board-certified epileptologist may review the video-EEG recordings to determine seizure type and electrographic onset and offset times. GTCS included tonic-clonic seizures of focal and generalized onset.

Data Processing, Statistics, and Machine Learning.

In some embodiments, differences in short recordings between patients with and without impending seizures may be analyzed. Group assignment could be different for individuals depending on seizure times, for example, a patient having a seizure at 11 p.m. would be in the seizure group for the evening recording but the without-seizure group for the morning recording, as no seizure was impending. In some embodiments, group differences of mean EDA, TEMP, HR, and RMS SD between the with seizure and without seizure groups may be analyzed (e.g., inclusion in seizure group determined by the patient having a seizure after 9 p.m. or after 6 a.m., for the two analyses respectively) via, for example, Mann-Whitney U tests. In some embodiments, for group classification, the seizure model engine 120 may implement one or more machine learning algorithms. Moreover, the machine learning algorithms may be evaluated for performance, such as each of seven machine learning algorithms: logistic regression (LR), k-nearest neighbor (KNN), random forest (RF), adaptive boosting (AB), Gaussian naive Bayes (GNB), and support vector machine (SVM) with linear or radial basis function kernel classifiers. In some embodiments, cross-validation may be used, such as, e.g., 10-fold cross-validation, in which hyperparameters are tuned by recursively by splitting the training dataset 10 times.

In some embodiments, for feature selection, the training and/or validation data sets may include a seizure during recording since the wristband was put on but before analysis time (e.g., time from wristband recording start to 9 p.m. or 6 a.m. respectively), EDA, HR, RMSSD, sex, age at first seizure, MRI findings, interictal generalized slowing (yes/no), interictal spikes (yes/no), and anti-seizure medication (ASM) reduction (yes/no). In some embodiments, model performance may be evaluated by randomly shuffling the data labels k times based on k-fold cross-validation and compare classifier performance of the shuffled and original labels, e.g., with a t-test. For LR, KNN, RF, AB, GNB, and SVM (linear and rbf) classifiers, providing patient-specific probability scores, performance may be evaluated with area under the curve of receiver operating characteristic (AUC-ROC) and with the Brier score, among other measures.

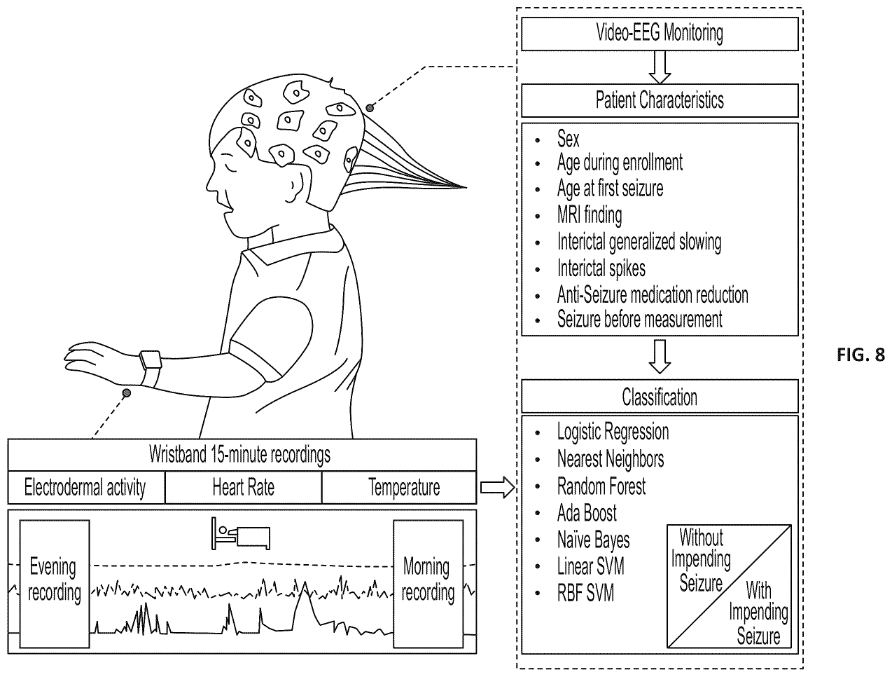

FIG. 8 depicts a schematic illustration of the experiment setup in a third example embodiment according to aspects of the present disclosure. Clinical and wearable data were collected from patients admitted to video-EEG monitoring and classified into recordings with and without impending seizures.

### Results

Patient Characteristics, Recordings, and Seizure Descriptions.

In some embodiments, an example data set may include 139 patients, in which 72 patients have no seizures and 67 patients have at least one seizure (GTCS or FIAS) during the concurrent video-EEG and wearable sensor data 102 recordings. For the morning and evening analyses, respective seizure groups may be determined by seizure occurrence time.

In the example, for the evening analysis, six patients may have at least one seizure before but no seizures after 9 p.m. and may thus be moved to the no-seizure group. Of the remaining 61 seizure patients, 42 patients may have no seizures before and at least one seizure after 9 p.m., and 19 patients may have at least one seizure before and after 9 p.m.

In the example, for the morning analysis, 22 patients of the evening data set may be excluded due to poor data quality, as determined by one or more data quality check. Of the remaining 118 total patients, 68 patients may have no seizures and 58 patients may have seizures. Eighteen patients may have at least one seizure before but no seizures after 6 a.m. and may thus be moved to the no-seizure group for the morning analysis. Of the remaining 38 seizure patients, 27 may have at least one seizure before and after 6 a.m., and 11 patients may have no seizures before and at least one seizure after 6 a.m.

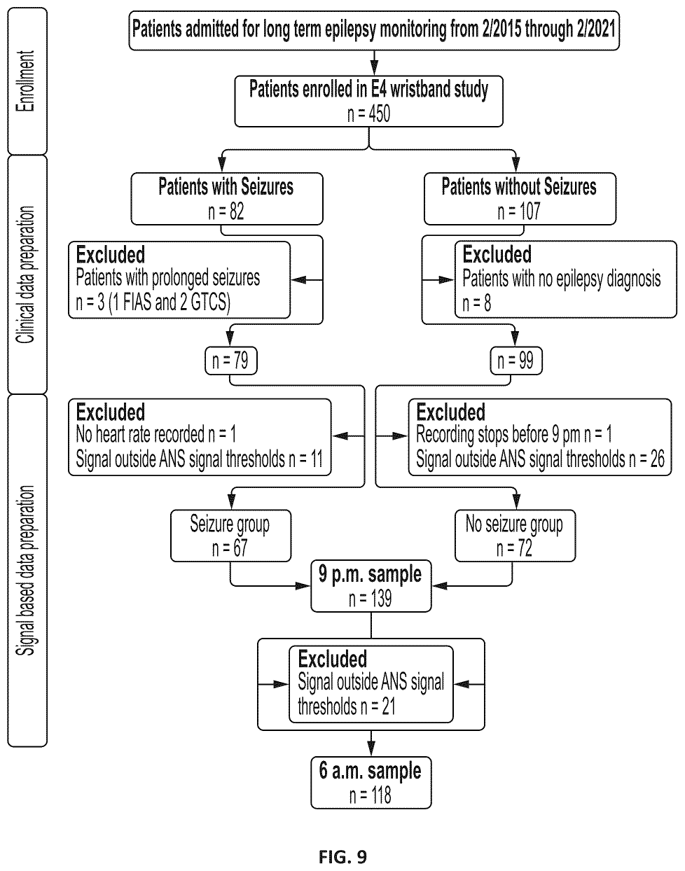

FIG. 9 depicts an inclusion diagram that visualizes the patient and signal selection procedures in a third example embodiment according to aspects of the present disclosure.

TABLE 8
Demographic and clinical characteristics of all patients included
in the statistical analysis.
Number of patients (total 139)

| | |
|---|---|
| With seizure | 67 |
| Without seizure | 72 |

Number of seizures (total 200)

| | |
|---|---|
| GTCS | 156 |
| FIAS | 37 |
| Other | 7 |

Sex

| | |
|---|---|
| Female | 65 |
| Male | 74 |

Age in years*

| | | |
|---|---|---|
| median (p25-p75) | 11.1 | (7.9-14.9) |

Age at first seizure*

| | | |
|---|---|---|
| median (p25-p75) | 4.0 | (1.3-8.6) |

Generalized slowing

| | |
|---|---|
| Yes | 121 |
| No | 18 |

Spikes

| | |
|---|---|
| Yes | 109 |
| No | 30 |

MRI finding

| | |
|---|---|
| Abnormal | 29 |
| Normal | 65 |
| Not available | 45 |

ASM reduction

| | |
|---|---|
| Yes | 55 |
| No | 78 |
| Not available | 6 |

*Due to high correlation between age and age at first seizure age is excluded for classification

Group Differences in Autonomic Markers.

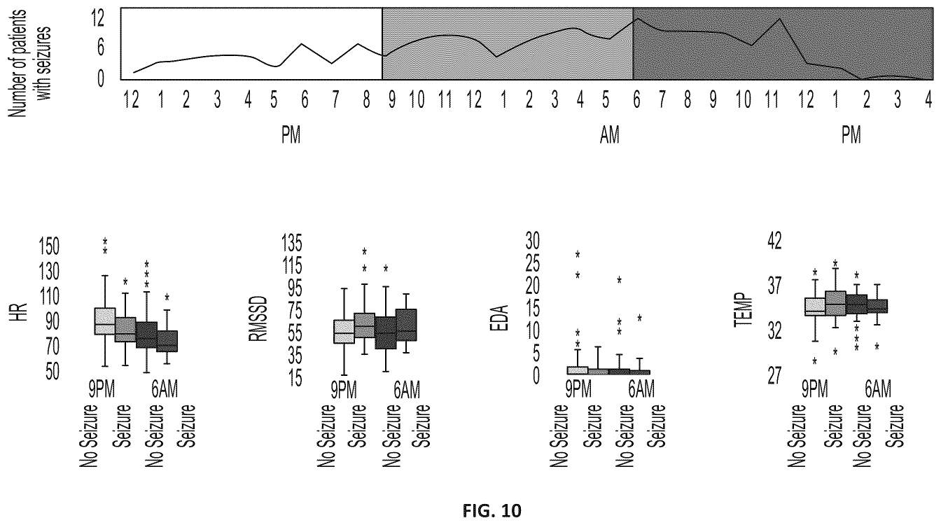

In some embodiments, to analyze group differences, seizure and no-seizure groups may be determined based on seizure occurrence time. Seizure patients may have at least one seizure after 9 p.m. or after 6 a.m. for the evening and morning analyses, respectively. An example of descriptive statistics are presented in FIG. 10.

FIG. 10 depicts a number of patients having a seizure at a time of the day in a third example embodiment according to aspects of the present disclosure. Vertical lines represent cutoffs for inclusion in the seizure after 9 p.m. and seizure after 6 a.m. groups for the evening and morning analysis, respectively. B illustrates box and whisker plots for the four biosensor modalities heart rate (HR), relative root mean square error (RMSSD) as a marker of heart rate variability, electrodermal activity (EDA), and temperature (TEMP) recorded at wrist or ankle.

In some embodiments, for patients with seizures, EDA (evening p<0.01) and HR (evening p=0.01) may be lower and RMSSD (evening p=0.02) may be higher compared to patients without seizure, while TEMP may not differ between groups and may therefore not be included in further analysis. In the example data set, for morning recordings, the group comparison is not significant (see Table 9 below for results).

TABLE 9
Group-wise descriptive statistics and Mann-Whitney U test results
for heart rate (HR), heart rate variability (RMSSD), electrodermal
activity (EDA), and peripheral body temperature (TEMP).

| | | | | | Percentiles | | | |
|---|---|---|---|---|---|---|---|---|
| | Group | Mean | Std | Median | 25 | 75 | U | p |
| | Time | 9:00 PM (n = 139) | | | | | | |
| HR | Without Sz | 93.91 | 18.80 | 90.25 | 80.61 | 102.77 | 1800 | 0.01 |
| | With Sz | 86.69 | 13.92 | 83.33 | 75.68 | 93.25 | | |
| RMSSD | Without Sz | 55.57 | 21.67 | 53.66 | 45.72 | 64.21 | 2938 | 0.02 |
| | With Sz | 62.29 | 19.46 | 59.62 | 53.24 | 71.14 | | |
| EDA | Without Sz | 2.67 | 5.51 | 0.36 | 0.17 | 1.29 | 1710 | <0.01 |
| | With Sz | 0.81 | 1.40 | 0.27 | 0.11 | 0.78 | | |
| TEMP | Without Sz | 35.14 | 1.90 | 35.30 | 34.02 | 36.64 | 2503 | 0.60 |
| | With Sz | 35.30 | 1.89 | 35.08 | 34.14 | 35.80 | | |
| | Time | 6:00 AM (n = 118) | | | | | | |
| HR | Without Sz | 82.47 | 19.88 | 81.67 | 69.57 | 92.82 | 1365 | 0.37 |
| | With Sz | 78.54 | 13.49 | 78.47 | 69.18 | 84.06 | | |
| RMSSD | Without Sz | 56.13 | 20.27 | 55.17 | 40.11 | 68.07 | 1773 | 0.15 |
| | With Sz | 61.57 | 17.82 | 58.84 | 47.95 | 75.49 | | |
| EDA | Without Sz | 1.72 | 3.93 | 0.46 | 0.22 | 1.26 | 1295 | 0.20 |
| | With Sz | 0.93 | 2.21 | 0.41 | 0.17 | 0.71 | | |
| TEMP | Without Sz | 35.22 | 1.44 | 35.57 | 34.30 | 36.13 | 1430 | 0.60 |
| | With Sz | 35.13 | 1.39 | 35.08 | 34.26 | 36.00 | | |

Classification Between Groups with and without an Impending Seizure.

Our classification models included sex, age at first seizure, MRI finding, interictal generalized slowing, interictal spikes, anti-seizure medication (ASM) reduction, seizure before measurement, as well as HR, RMSSD, and EDA (see Table 10 below for statistics). For the evening and morning analyses respectively, accuracy averaged over all classifiers was 69% and 76%, and mean AU-ROC was 0.73 and 0.80. Testing accuracies against random shuffle runs revealed significantly better performance for true labels. Averaged Brier score for probabilistic forecasts of seizure occurrence was 0.21 and 0.18 for the evening and morning time windows, respectively. To summarize, a 15-minute ANS recording contains information that contributes to the differentiation between patients with and without an impending seizure.

TABLE 10
Classifier performance for the distinction between
recordings of patients with and without seizures.

| | | | | | | | |
|---|---|---|---|---|---|---|---|
| | | | | Brier | | Shuffle | P- |
| Classifier | Accuracy | Sensitivity | Specificity | score | AUC_ROC | Accuracy (SD) | value |

9:00 PM

| | | | | | | | |
|---|---|---|---|---|---|---|---|
| Logistic | 0.70 | 0.59 | 0.78 | 0.20 | 0.77 | 0.51 (0.039) | <0.001 |
| Regression | | | | | | | |
| Nearest | 0.72 | 0.66 | 0.76 | 0.21 | 0.73 | 0.53 (0.039) | <0.001 |
| Neighbors | | | | | | | |
| Random | 0.65 | 0.57 | 0.78 | 0.23 | 0.69 | 0.52 (0.047) | <0.001 |
| Forest | | | | | | | |
| Ada Boost | 0.63 | 0.21 | 0.93 | 0.24 | 0.60 | 0.55 (0.034) | <0.001 |
| Naive | 0.69 | 0.61 | 0.76 | 0.21 | 0.77 | 0.54 (0.042) | <0.001 |
| Bayes | | | | | | | |
| Linear | 0.72 | 0.62 | 0.80 | 0.20 | 0.79 | 0.54 (0.034) | <0.001 |
| SVM | | | | | | | |
| RBF SVM | 0.69 | 0.59 | 0.76 | 0.19 | 0.79 | 0.54 (0.036) | <0.001 |

6:00 AM

| | | | | | | | |
|---|---|---|---|---|---|---|---|
| Logistic | 0.78 | 0.58 | 0.87 | 0.16 | 0.81 | 0.65 (0.015) | <0.001 |
| Regression | | | | | | | |
| Nearest | 0.71 | 0.45 | 0.82 | 0.19 | 0.78 | 0.63 (0.031) | <0.001 |
| Neighbors | | | | | | | |
| Random | 0.77 | 0.53 | 0.89 | 0.18 | 0.81 | 0.62 (0.026) | <0.001 |
| Forest | | | | | | | |
| Ada Boost | 0.76 | 0.71 | 0.78 | 0.18 | 0.74 | 0.69 (0.009) | <0.001 |
| Naive | 0.75 | 0.32 | 0.94 | 0.18 | 0.81 | 0.68 (0.021) | <0.001 |
| Bayes | | | | | | | |
| Linear | 0.74 | 0.55 | 0.82 | 0.18 | 0.79 | 0.67 (0.014) | <0.001 |
| SVM | | | | | | | |
| RBF SVM | 0.79 | 0.05 | 0.92 | 0.16 | 0.85 | 0.68 (0.019) | <0.001 |

### Discussion

Summary.

In some embodiments, a seizure risk assessment can be obtained from short-term ANS recordings and clinical data. This approach has the potential to inform inpatient clinical decisions and outpatient seizure monitoring systems.

Electrodermal and cardiac activity signals differ in evening short-term recordings between patients with or without impending seizures. HR measures are normally compared between people with epilepsy and controls, showing higher HR for epilepsy patients. Also, HR patterns differ between seizure types and time of day. In some embodiments, within a group of patients diagnosed with epilepsy, patients with an impending seizure may exhibit lower HR and higher heart rate variability (HRV) compared to patients with no seizure in evening recordings. Accordingly, the wearable sensor 101 may pick-up seizure-related changes in cardiac activity before the seizures occur. Additionally, in some embodiments, patients with an impending seizure may have lower EDA levels compared to those without impending seizures, and thus the wearable sensor data 102 and/or seizure recordings show lower amplitude in 24-hour EDA curves and lower pre-ictal baselines compared to time-matched expected interictal values. In some embodiments, the lower levels of ANS activity before a seizure may be related to lower adaptability and stressor processing in the ANS. In some embodiments, the evening recording may inform night monitoring and could thereby become a useful tool in sudden unexplained death in epilepsy (SUDEP) prevention.

The Seizure Forecasting Model Combines Multimodal Autonomic Activity Markers with Clinical Variables.

In some embodiments, seizure forecasting by the seizure model engine 120 may include a multimodal approach. Applying machine learning tools to short (e.g., 5, 10, or 15-minute) multimodal recordings of autonomic activity enables prediction of seizure probability in a next period better than chance. In some embodiments, additional features could be derived from the ANS recordings or that additional clinical variables could be included for training and/or as input features to the machine learning algorithm. For example, patients could be asked to answer daily questions regarding medication intake, seizure occurrence, or seizure characteristics. Seizure diaries may also improve the model, as they indicate seizure cycles and frequency, potential seizure triggers, auras, and perceived seizure likelihood.

Seizure Risk Assessment from Short Evening and Morning ANS Recordings Complements Existing Approaches.

Accordingly, in some embodiments, a patient may wear the wearable sensor 101, which may take a 15-minute recording of wearable sensor data 102 during a rest period before sleep and after waking up. In some embodiments, the patient additionally answer questions, such as whether they had a seizure that day. In some embodiments, the seizure monitoring system 110 may monitor and extend the recording to ensure each recording contains a full 15 minutes of high-quality data.

In some embodiments, the recording time (e.g., bedtime) can reveal important information about the impact of seizures on daily life. At the beginning of monitoring, there may be no individual comparison data, such as environmental, medical, or social changes that would affect wearing a device continuously. In this case, a short-term measurement could be run in combination with seizure prediction algorithms of the seizure model engine 120 based on continuous data to determine when the wearable sensor 101 should be worn (e.g., overnight or for the following day). In some embodiments, the wearable sensor data 102 may be combined with forecasting and prediction tools based on EEG, intercranial EEG, or seizure diary data.

Moreover, in some embodiments, potential mechanisms of SUDEP may involve autonomic dysfunctions, and night-time supervision has the potential to reduce SUDEP risk. As a result, in some embodiments, the wearable sensor data 102 recordings taken from the wearable sensor 101 in the evening when SUDEP risk is high may be used to determine predictive and preventative SUDEP biomarkers.

Forecast Accuracy.

In some embodiments, cardiac and electrodermal data collected from the wearable sensor 101, alone or together with knowledge of prior seizures that day, can be used to improve the seizure likelihood forecast over random chance. Additional physiological data from the wearable sensor 101 and/or from other sensors, or additional clinical data, may improve the accuracy of the predictions. Seizure prediction is extremely relevant, specifically in patients with more severe seizures, complications from seizures, status epilepticus, or apnea during seizures. Therefore, individual patient circumstances may be considered to determine which additional physiological or clinical data is necessary. Furthermore, input features and cutoffs can be adjusted to the individual patient, e.g., time of recording based on seizure type-specific occurrence time, and according to the clinical purpose. For example, a higher false alarm rate is tolerable if the decision is to continuously monitor ANS activity throughout the day, while for planning a hospital stay a high specificity is desired.

Short-Term Measurements are Useful in Other Health-Related Assessments.

In some embodiments, the use of ANS data including HR, resting HR, HR variability, EDA, TEMP, blood pressure, etc. may have multiple applications in global health state assessment, which might lead to low specificity. In the context of epilepsy, rest measures can be used to determine seizure likelihood, load recommendations or ASM dosage for the day. Thus, selecting the appropriate modalities and clinical information may facilitate seizure forecasting model success.

In some embodiments, seizure likelihood prediction may be performed using brief resting autonomic measurements from wearable sensors 101. In some embodiments, in the inpatient setting, ANS data may be recorded simultaneously with video-EEG. For example, concurrent video-EEG and ANS recordings may inform the length of EMU stay. In some embodiments, measurement times, e.g., timing of the short recording, may be adjusted based on seizure probability determinants, such as vigilance state, or seizure type. In some embodiments, a data quality checking system may be employed to monitor the 15-minute recordings. For example, the recordings would be monitored online (e.g., by the seizure monitoring system 110) and stopped when a full 15-minutes of high-quality data is reached. There are currently no uniform data quality standards for ANS recordings from wearables. In some embodiments, a short-term analysis on a longitudinal basis for individual patients may be helpful by combining short recordings with other seizure likelihood assessment tools, such as diaries.

### CONCLUSION

In some embodiments, short-term autonomic recordings from a wearable sensor 101 may enable future seizure likelihood assessments. Estimating seizure risk based on brief high-quality recordings may accompany long-term video-EEG recordings or may be used when a patient's wearable sensor 101 is forgotten or unavailable. The practicality and the prospect of higher patient compliance illustrate the benefits of the approach to develop a seizure prediction system based on regularly recorded short data segments. In some embodiments, this approach offers the potential to develop an out-of-the-box cross-sectional analysis that can be specified with individual profiles over time, accounting for common patterns and the high individuality of seizure generation. Seizure forecast accuracy may be improved further by incorporating additional physiological, clinical, or personalized data into our approach.

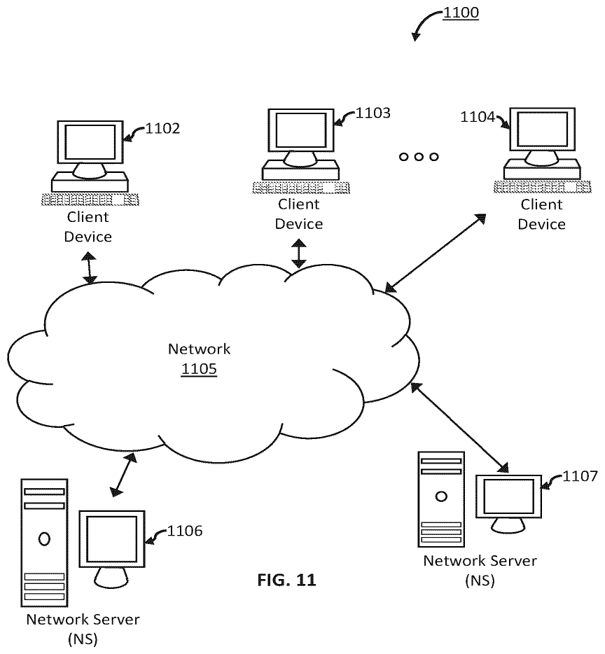

FIG. 11 depicts a block diagram of an exemplary computer-based system and platform 1100 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 1100 may be configured to manage a large number of members and concurrent transactions, as detailed herein. In some embodiments, the exemplary computer-based system and platform 1100 may be based on a scalable computer and network architecture that incorporates varies strategies for assessing the data, caching, searching, and/or database connection pooling. An example of the scalable architecture may be an architecture that may be capable of operating multiple servers.

In some embodiments, referring to FIG. 11, member computing device 1102, member computing device 1103 through member computing device 1104 (e.g., clients) of the exemplary computer-based system and platform 1100 may include virtually any computing device capable of receiving and sending a message over a network (e.g., cloud network), such as network 1105, to and from another computing device, such as servers 1106 and 1107, each other, and the like. In some embodiments, the member devices 1102-1104 may be personal computers, multiprocessor systems, microprocessor-based or programmable consumer electronics, network PCs, and the like. In some embodiments, one or more member devices within member devices 1102-1104 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 1102-1104 may be devices that may be capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that may be equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In some embodiments, one or more member devices within member devices 1102-1104 may include may run one or more applications, such as Internet browsers, mobile applications, voice calls, video games, videoconferencing, and email, among others. In some embodiments, one or more member devices within member devices 1102-1104 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 1102-1104 may be specifically programmed by either Java, .Net, QT, C, C++ and/or other suitable programming language. In some embodiments, one or more member devices within member devices 1102-1104 may be specifically programmed include or execute an application to perform a variety of possible tasks, such as, without limitation, messaging functionality, browsing, searching, playing, streaming, or displaying various forms of content, including locally stored or uploaded messages, images and/or video, and/or games.

In some embodiments, the exemplary network 1105 may provide network access, data transport and/or other services to any computing device coupled to it. In some embodiments, the exemplary network 1105 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 1105 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 1105 may include and implement, as an alternative or in conjunction with one or more of the above, a WiMAX architecture defined by the WiMAX forum. In some embodiments and, optionally, in combination of any embodiment described above or below, the exemplary network 1105 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 1105 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 1105 may also include mass storage, such as network attached storage (NAS), a storage area network (SAN), a content delivery network (CDN) or other forms of computer or machine readable media.

In some embodiments, the exemplary server 1106 or the exemplary server 1107 may be a web server (or a series of servers) running a network operating system, examples of which may include but are not limited to Microsoft Windows Server, Novell NetWare, or Linux. In some embodiments, the exemplary server 1106 or the exemplary server 1107 may be used for and/or provide cloud and/or network computing. Although not shown in FIG. 11, in some embodiments, the exemplary server 1106 or the exemplary server 1107 may have connections to external systems like email, SMS messaging, text messaging, ad content providers, etc. Any of the features of the exemplary server 1106 may be also implemented in the exemplary server 1107 and vice versa.

In some embodiments, one or more of the exemplary servers 1106 and 1107 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 1101-1104.

In some embodiments and, optionally, in combination of any embodiment described above or below, for example, one or more exemplary computing member devices 1102-1104, the exemplary server 1106, and/or the exemplary server 1107 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

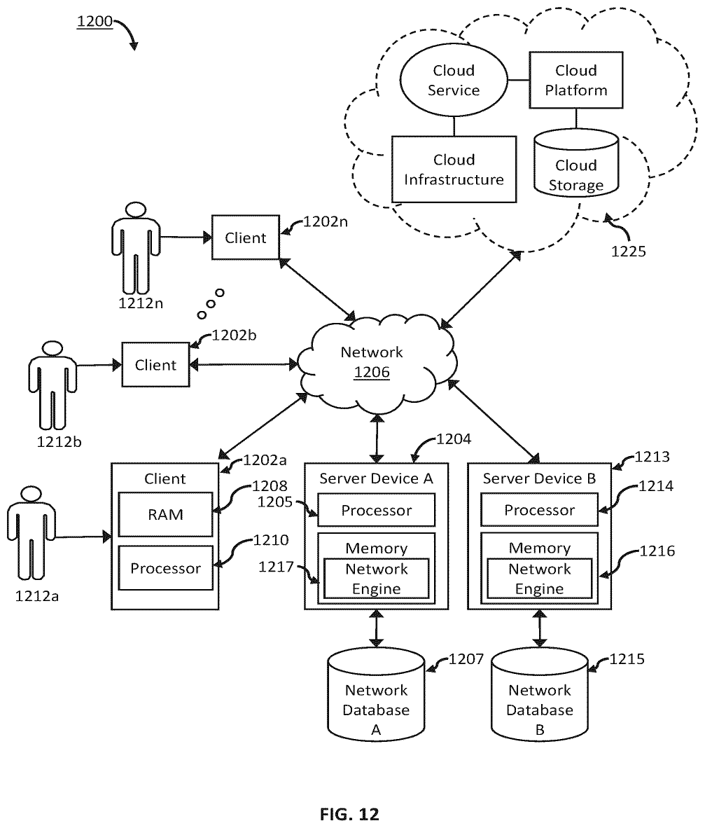

FIG. 12 depicts a block diagram of another exemplary computer-based system and platform 1200 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing device 1202a, member computing device 1202b through member computing device 1202n shown each at least includes a computer-readable medium, such as a random-access memory (RAM) 1208 coupled to a processor 1210 or FLASH memory. In some embodiments, the processor 1210 may execute computer-executable program instructions stored in memory 1208. In some embodiments, the processor 1210 may include a microprocessor, an ASIC, and/or a state machine. In some embodiments, the processor 1210 may include, or may be in communication with, media, for example computer-readable media, which stores instructions that, when executed by the processor 1210, may cause the processor 1210 to perform one or more steps described herein. In some embodiments, examples of computer-readable media may include, but are not limited to, an electronic, optical, magnetic, or other storage or transmission device capable of providing a processor, such as the processor 1210 of client 1202a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, Matlab, and etc.

In some embodiments, member computing devices 1202a through 1202n may also comprise a number of external or internal devices such as a mouse, a CD-ROM, DVD, a physical or virtual keyboard, a display, or other input or output devices. In some embodiments, examples of member computing devices 1202a through 1202n (e.g., clients) may be any type of processor-based platforms that are connected to a network 1206 such as, without limitation, personal computers, digital assistants, personal digital assistants, smart phones, pagers, digital tablets, laptop computers, Internet appliances, and other processor-based devices. In some embodiments, member computing devices 1202a through 1202n may be specifically programmed with one or more application programs in accordance with one or more principles/methodologies detailed herein. In some embodiments, member computing devices 1202a through 1202n may operate on any operating system capable of supporting a browser or browser-enabled application, such as Microsoft™, Windows™, and/or Linux. In some embodiments, member computing devices 1202a through 1202n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 1202a through 1202n, user 1212a, user 1212b through user 1212n, may communicate over the exemplary network 1206 with each other and/or with other systems and/or devices coupled to the network 1206. As shown in FIG. 12, exemplary server devices 1204 and 1213 may include processor 1205 and processor 1214, respectively, as well as memory 1217 and memory 1216, respectively. In some embodiments, the server devices 1204 and 1213 may be also coupled to the network 1206. In some embodiments, one or more member computing devices 1202a through 1202n may be mobile clients.

In some embodiments, at least one database of exemplary databases 1207 and 1215 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that may be stored.

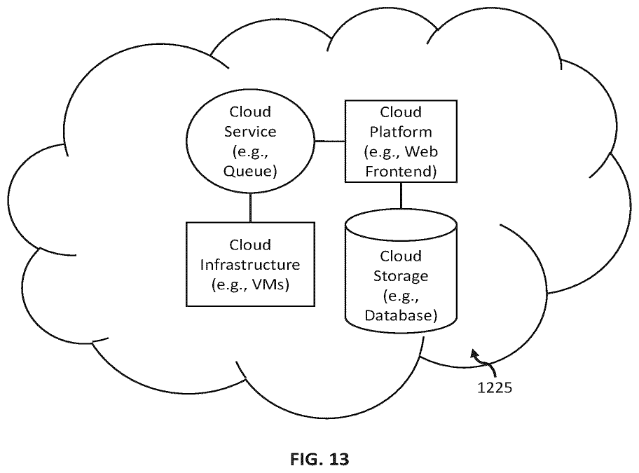

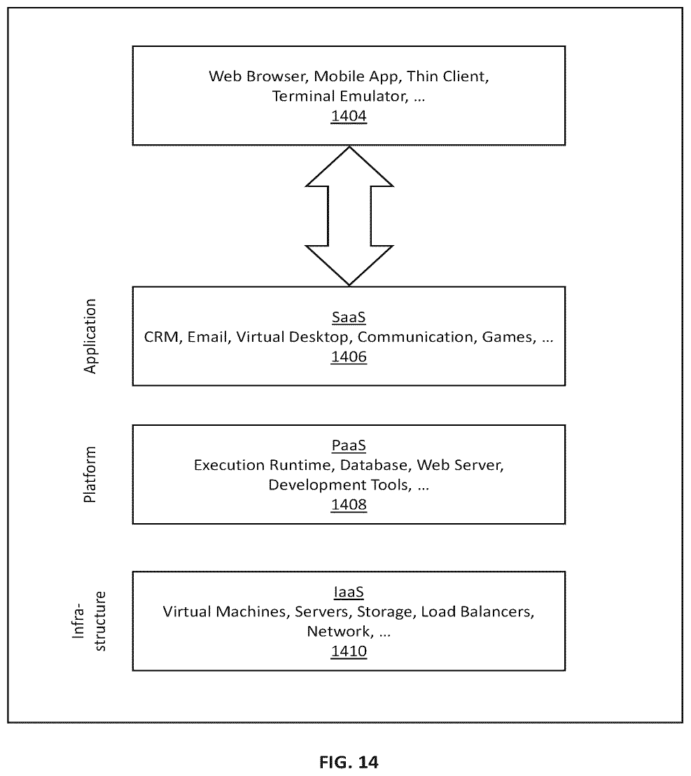

In some embodiments, the exemplary inventive computer-based systems/platforms, the exemplary inventive computer-based devices, and/or the exemplary inventive computer-based components of the present disclosure may be specifically configured to operate in a cloud computing/architecture 1225 such as, but not limiting to: infrastructure a service (IaaS) 1410, platform as a service (PaaS) 1408, and/or software as a service (SaaS) 1406 using a web browser, mobile app, thin client, terminal emulator or other endpoint 1404. FIGS. 13 and 14 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) Linux, (2) Microsoft Windows, (3) OS X (Mac OS), (4) Solaris, (5) UNIX (6) VMWare, (7) Android, (8) Java Platforms, (9) Open Web Platform, (10) Kubernetes or other suitable computer platforms. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but may be not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

In some embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RCS, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

While one or more embodiments of the present disclosure have been described, it may be understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

1. A method comprising:
receiving, by at least one processor, at least one data stream comprising wearable sensor data associated with a user;
wherein the at least one data stream comprises electrodermal activity data;
wherein the electrodermal activity data comprises circadian rhythm-dependent amplitudes;
receiving, by the at least one processor, at least one time span associated with at least one seizure of the user; and
training, by the at least one processor, seizure machine learning model to identify a pre-ictal period associated with a time segment based at least in part on the circadian rhythm dependent amplitudes and the at least one time span associated with the at least one seizure.

2. The method as recited in claim 1, further comprising:
receiving, by the at least one processor, at least one subsequent data stream comprising additional wearable sensor data;
utilizing, by the at least one processor, the seizure machine learning model to identify at least one pre-ictal period based at least in part on the at least on subsequent data stream; and
generating, by the at least one processor, a seizure alert on a computing device associated with the user to alert the user of an impending seizure.

3. The method as recited in claim 2, further comprising communicating, by the at least one processor, with a wearable device to receive at least one subsequent data stream in real-time.

4. The method as recited in claim 3, wherein the wearable device includes a biomarker sensor worn by the user.

5. The method as recited in claim 1, wherein the time segment used to calculate forecasts comprises twenty-four hours.

6. The method as recited in claim 2, further comprising determining, by the at least one processor, an inter-ictal period upon the pre-ictal period probability falling below the pre-ictal probability threshold.

7. The method as recited in claim 6, further comprising maintaining, by the at least one processor, an alert status associated with the pre-ictal alert until a seizure occurrence period has passed.

8. The method as recited in claim 7, wherein the seizure occurrence period comprises one hour.

9. A system comprising:
at least one sensor; and
at least one processor in communication with the at least one sensor and configured to perform steps of instructions stored in a non-transitory memory, the steps comprising:
receiving, by at least one processor, at least one data stream comprising wearable sensor data associated with a user;
wherein the at least one data stream comprises electrodermal activity data;
wherein the electrodermal activity data comprises circadian rhythm-dependent amplitudes;
receiving, by the at least one processor, at least one time span associated with at least one seizure of the user; and
training, by the at least one processor, seizure machine learning model to identify a pre-ictal period associated with a time segment based at least in part on the circadian rhythm dependent amplitudes and the at least one time span associated with the at least one seizure.

10. The system as recited in claim 9, wherein the at least one processor may be further configured to:
receive at least one subsequent data stream comprising additional wearable sensor data;
utilize the seizure machine learning model to identify at least one pre-ictal period based at least in part on the at least on subsequent data stream; and
generate a seizure alert on a computing device associated with the user to alert the user of an impending seizure.

11. The system as recited in claim 10, wherein the at least one processor may be further configured to communicate with a wearable device to receive the at least one subsequent data stream in real-time.

12. The system as recited in claim 11, wherein the wearable device includes a biomarker sensor worn by the user.

13. The system as recited in claim 9, wherein the time segment used to calculate forecasts comprises twenty-four hours.

14. The system as recited in claim 10, wherein the at least one processor may be further configured to determine an inter-ictal period upon the pre-ictal period probability falling below the pre-ictal probability threshold.

15. The system as recited in claim 14, wherein the at least one processor may be further configured to maintain an alert status associated with the pre-ictal alert until a seizure occurrence period has passed.

16. The system as recited in claim 15, wherein the seizure occurrence period comprises one hour.
